(19) United States
(12) Patent Application Publication
(10) Pub. No.: US 2009/0221019 A1
(43) Pub. Date: Sep. 3, 2009

(54) Core-Modified Terpene Trilactones From Ginkgo Biloba Extract and Biological Evaluation Thereof

(76) Inventors: Koji Nakanishi, New York, NY (US); Sergei Dzyuba, New York, NY (US); Hideki Ishii, Gifu-ken (JP)

Correspondence Address: COOPER & DUNHAM, LLP, 30 Rockefeller Plaza, 20th Floor, NEW YORK, NY, 10112, US

(21) Appl. No.: 11/922,903

(22) PCT Filed: Jun. 22, 2006
(86) PCT No.: PCT/US2006/024492

§ 371 Date: Apr. 16, 2009

Related U.S. Application Data
(60) Provisional application No. 60/693,228, filed on Jun. 22, 2005.
(60) Provisional application No. 60/715,871, filed on Sep. 9, 2005.

(51) Int. Cl. C12Q 1/02; C07D 493/22

(52) U.S. Cl. 435/29; 549/265

(57) ABSTRACT

Lactone-rings of ginkgolides are converted into the corresponding tetrahydrofuran moieties via DIBAL-H reduction followed by deoxygenation of the formed lactols with Et₃SiH/BF₃Et₂O producing a series of lactol-free ginkgolides. The present invention also relates to synthesis of hydroxyl-free, or hydroxyl-free and lactone-free, ginkgolides and bilobalides.

This application claims benefit of U.S. Provisional Application No. 60/693,228, filed Jun. 22, 2005 and of U.S. Provisional Application No. 60/715,871, filed Sep. 9, 2005, the contents of each of which are hereby incorporated by reference.

The invention disclosed herein was made with Government support under grant no. GM-MHO68817 from the National Institutes of Health. Accordingly, the U.S. Government has certain rights in this invention.

Throughout this application, various publications are referenced by number in parentheses. The disclosures of these publications in their entireties are hereby incorporated by reference into this application in order to more fully describe the state of the art as known to those skilled therein as of the date of the invention described and claimed herein.

### BACKGROUND OF THE INVENTION

Terpene trilactones (FIG. 1), the main active ingredients of the Ginkgo biloba extract, have attracted a lot of attention over the years due to their unique biological properties. Recently, Ginkgo biloba extract and ginkgolides where shown to suppress the progression of the Alzheimer's disease, via a variety of potential pathways (2).

We have demonstrated here in a series of electrophysiological experiments that several native ginkgolides are capable of protecting hippocampal neuronal cell cultures from the β-amyloid induced impairment of long-term potentiation, and of cell death. Also a synthetic, more hydrophobic, derivative of ginkgolide A, in which all lactone moieties have been converted into tetrahydrofuran moieties, so-called ginkgolide A “Triether”, (3), is active in this role.

An increased hydrophobicity of “GA-triether”, or of any terpene trilactone cage skeleton, should facilitate its cell wall permeability, thus making it a more viable candidate than native ginkgolides. Moreover, from the synthetic stand-point of view, ginkgolide's cage-like skeleton, with three lactone rings of different reactivities towards reducing agents, creates an attractive scaffold to conduct lactone to ether reduction.

We also disclose here dehydroxylation of natural ginkgolides, and synthetic routes for making lactol- and hydroxyl-free ginkgolides, so-called “naked” ginkgolides. The increased hydrophobicity of the partial and fully naked ginkgolides is expected to promote their ability to cross the blood brain barrier and thus enhance their potential in central nervous system disorders such as Alzheimer's disease.

Further disclosed are related methods for functionalizing ginkgolides and derivatives at their hydroxyl moieties.

### SUMMARY OF THE INVENTION

In one embodiment this invention provides process of reducing a lactone or of replacing or removing a hydroxyl group on a terpene trilactone cage skeleton or a bilobalide comprising:
a) obtaining a lactone bearing terpene trilactone cage skeleton or bilobalide, or a hydroxyl bearing terpene trilactone cage skeleton or bilobalide, and
b) (i) exposing the lactone bearing terpene trilactone cage skeleton or bilobalide to DIBAL-H in a first suitable solvent to reduce the lactone and form a resulting compound having a hydroxyl group at the position of the lactone; or
   (ii) exposing the hydroxyl bearing terpene trilactone cage skeleton or bilobalide to an alkylating agent capable of undergoing a subsequent deoxygenation, in the presence of DMAP and a second suitable solvent to form a first product and exposing the first product to Et₃SiH and Bz₂0 in the presence of a third suitable solvent or to Bu₃SnH and AlBN in the presence of a fourth suitable solvent, or exposing the hydroxyl bearing terpene trilactone cage skeleton or bilobalide to Et₃SiH and BF₃-Et₂0 in the presence of a fifth suitable solvent for a time sufficient to deoxygenate the hydroxyl group, or exposing the hydroxyl bearing terpene trilactone cage skeleton or bilobalide to an allylating agent and TiCl₄ or BF₃-Et₂0 in the presence of a seventh suitable solvent, so as to thereby replace the hydroxyl group on the terpene trilactone cage skeleton or bilobalide; or
   (iii) exposing the hydroxyl bearing terpene trilactone cage skeleton or bilobalide to (diethylamino)sulfur trifluoride and pyridine in the presence of a sixth suitable solvent for a time sufficient to remove the hydroxyl group.

### BRIEF DESCRIPTION OF THE FIGURES

FIG. 1: Naturally occurring terpene trilactones ginkgolides A, C, B, J, the GA-triether, and bilobalide.

FIG. 2: A synthesis of GA Triether.

FIG. 3: Step-wise conversion of GA into “GA-triether”. Conditions: (a) DIBAL-H (5 eq), THF, −78° C., 2 h; H⁺-work-up; (b) Et₃SiH, BF₃-ether, CH₂Cl₂, −78° C. to room temperature (rt), 12 h.

FIG. 4: Direct synthesis of GA Triether. Conditions: (a) DIBAL-H, (24 eq.), THF, −78° C., 2 h; (b) Et₃SiH, BF₃-ether, CH₂Cl₂, −78° C. to rt, 12 h.

FIG. 5: Permethylation of GA and GA-triether; conditions: (a) MeI (10 eq.), AgOTf, Et₃N, THF reflux; (b) MeI (50 eq.), KH, THF, rt.

FIG. 6: Reduction of dimethyl-GA. Conditions: (a) DIBAL-H (4.5 eq), THF, −78° C., 2 h; H⁺-work-up.

FIG. 7: Reduction of GB. Conditions: (a) DIBAL-H (4.5 eq), THF, −78° C., 2 h; H⁺-work-up; (b) Et₃SiH, BF₃-ether, CH₂Cl₂, −78° C. to rt, 12 h.

FIG. 8: Attempted direct synthesis of “GB-triether”. conditions: (a) DIBAL-H (4.5 eq), THF, 78° C., 2 h; H⁺-work-up; (b) Et₃SiH, BF₃-ether, CH₂Cl₂, −78° C. to rt, 12 h.

FIGS. 9A, 9B & 9C: (9A) Shows synthesis of GJ from GC; (9B) shows deoxygenation of a ginkgolide A hydroxyl; and (9C) shows synthesis of a “naked” ginkgolide.

FIG. 10: Aβ-induced LTP impairment in the CA1 region of hippocampal slices and its reversal by P8A. The horizontal bar and the arrows indicate a 20 min period during which Aβ and/or P8A were added to the bath solution and the time at which the theta-burst stimulation was applied, respectively. Every fourth recording point is shown for clarity.

FIGS. 11A and 11B: Effect of individual ginkgolides and bilobalide on Aβ-induced LTP impairment in CA1 region of hippocampal slices. Experiments in A (active compounds) and B (inactive compounds) were interleaved with each other; the horizontal bar and the arrows indicate a 20 min period during which Aβ and/or ginkgolides were added to the bath solution and the time at which the theta-burst stimulation was applied, respectively. Every fourth recording point is shown for clarity.

FIG. 12. Residual potentiation at the end of the recording, 155 min.

FIG. 13. Effect of P8A, GA and GJ on the survival of cultures hippocampal neurons treated with oligomeric Aβ peptide. Student-Newman-Keuls multiple comparison test, p<0.01 for Aβ vs TTL+Aβ, *, p<0.05 for Aβ vs GJ+Aβ, **.

FIG. 14: Removal of lactones and methylation of ginkgolides.

FIG. 15: Synthesis of GA and GB “triethers”. Condition a) is DIBAL-H, THF, 2 h; b) is Et₃SiH, BF₃-Et₂O, CH₂Cl₂, 12 h.

FIG. 16: Synthesis of GC and GJ “triethers”. Condition a) is DIBAL-H, THF, 2 h; b) is Et₃SiH, BF₃-Et₂O, CH₂Cl₂, 12 h.

FIG. 17A: (A) Synthesis of hydroxyl-free ginkgolides from GC.

FIG. 18A: (A) Synthesis of hydroxyl-free ginkgolides from GA.

FIG. 19: Various GC, GB and GA lactone-free ginkgolides.

FIG. 20: Selective functionalization of ginkgolide C.

FIG. 21: Selective multiple simultaneous functionalization of ginkgolide C.

FIG. 22: Regioselelective removal of hydroxyl groups via two-step thiocarbonylation/deoxygenation process.

FIG. 23: Theoretical Dehydration of OH-3 from unprotected GC to create predicted intermediate 1 in en route to efficient synthesis of GM.

FIG. 24: Treatment of GA with (diethylamino)sulfur trifluoride (DAST) provides no fluorodehydroxylation at the C-10 position, but instead leads to a high yield elective elimination of the tertiary hydroxy group, OH-3, affording ginkgolide L (GL).

FIG. 25: Reaction of 10-benzyl-GC 4 with DAST in the presence of pyridine in THF results in a clean elimination of the OH-3 group giving unsaturated lactone 5 in good yield.

FIG. 26: Lactones can be removed form the terpene trilactone cage skeleton or bilobalide using Et₃Siallyl.

FIG. 27: Scheme for functionalizing a ginkgolide at the C10 position; ginkgolide B exemplified.

FIG. 28: Scheme for functionalizing a ginkgolide at the C10 position; ginkgolide C exemplified.

FIG. 29: Scheme for functionalizing ginkgolide C at the C7 position.

### DETAILED DESCRIPTION

This invention provides a process of reducing a lactone or of replacing or removing a hydroxyl group on a terpene trilactone cage skeleton or a bilobalide comprising:
a) obtaining a lactone bearing terpene trilactone cage skeleton or bilobalide, or a hydroxyl bearing terpene trilactone cage skeleton or bilobalide, and
b) (i) exposing the lactone bearing terpene trilactone cage skeleton or bilobalide to DIBAL-H in a first suitable solvent to reduce the lactone and form a resulting compound having a hydroxyl group at the position of the lactone; or
   (ii) exposing the hydroxyl bearing terpene trilactone cage skeleton or bilobalide to an alkylating agent capable of undergoing a subsequent deoxygenation, in the presence of DMAP and a second suitable solvent to form a first product and exposing the first product to Et₃SiH and Bz₂0 in the presence of a third suitable solvent or to Bu₃SnH and AlBN in the presence of a fourth suitable solvent, or exposing the hydroxyl bearing terpene trilactone cage skeleton or bilobalide to Et₃SiH and BF₃-Et₂0 in the presence of a fifth suitable solvent for a time sufficient to deoxygenate the hydroxyl group, or exposing the hydroxyl bearing terpene trilactone cage skeleton or bilobalide to an allylating agent and TiCl₄ or BF₃-Et₂0 in the presence of a seventh suitable solvent, so as to thereby replace the hydroxyl group on the terpene trilactone cage skeleton or bilobalide; or
   (iii) exposing the hydroxyl bearing terpene trilactone cage skeleton or bilobalide to (diethylamino)sulfur trifluoride and pyridine in the presence of a sixth suitable solvent for a time sufficient to remove the hydroxyl group.

DIBAL-H may be substituted with Red-Al or with a borane.

This invention further provides the instant process, wherein the lactone bearing terpene trilactone cage skeleton is ginkgolide A, ginkgolide B, ginkgolide C, ginkgolide J, or ginkgolide M.

This invention provides the instant process for reducing a lactone of a lactone bearing terpene trilactone cage skeleton or bilobalide wherein in the process the lactone is reduced by exposing the lactone bearing terpene trilactone cage skeleton or bilobalide to DIBAL-H in a first suitable solvent to form a resulting compound having a hydroxyl group at the position of the lactone.

This invention further provides the instant process for replacing a hydroxyl group on a terpene trilactone cage skeleton or a bilobalide, wherein in the process the hydroxyl bearing terpene trilactone cage skeleton is exposed to the alkylating agent capable of undergoing a subsequent deoxygenation, in the presence of DMAP and the second suitable solvent to form the first product, and the first product is exposed to Et₃SiH and Bz₂0 in the presence of the third suitable solvent or to Bu₃SnH and AlBN in the presence of the fourth suitable solvent, so as to remove the hydroxyl group.

This invention further provides the instant process for replacing a hydroxyl group on a terpene trilactone cage skeleton or a bilobalide, wherein in the process the hydroxyl bearing terpene trilactone cage skeleton is exposed to Et₃SiH and BF₃-Et₂0 in the presence of the fifth suitable solvent for the time sufficient to deoxygenate the hydroxyl group at the position of the lactone so as to thereby remove the hydroxyl group.

This invention further provides the instant process, wherein the alkylating agent has the structure:

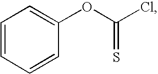

### or an RBr or an RCl.

This invention further provides the instant process, wherein the first suitable solvent and/or fifth suitable solvent is THF. This invention further provides the instant process, wherein the first suitable solvent is THF/Hexane. This invention further provides the instant process, wherein the second suitable solvent is CH₃CN or DMF. This invention further provides the instant process, wherein the third suitable solvent and/or fourth suitable solvent is toluene or CH₂Cl₂. This invention further provides the instant process wherein the first and/or fifth solvent is dichloromethane (CH₂Cl₂) or dioxane. This invention further provides the second suitable solvent is THF, dichloromethane (CH₂Cl₂) or dioxane. This invention further provides the third and/or fourth suitable solvent wherein the solvent is benzene, chloroform, THF.

This invention further provides the instant process, wherein step b) (i) or b) (ii) is performed at a temperature of 20 to 30° C. This invention further provides the instant process wherein step b)(i) or b) (ii) is performed at a temperature of about 25° C. This invention further provides the instant process, wherein step a) is performed at a temperature of −70° C. to −80° C. This invention further provides the instant process, wherein step a) is performed at a temperature of about −75° C.

This invention further provides the instant process, wherein in step b) (i) 4-5 equivalents of DIBAL-H are employed. This invention further provides the instant process, wherein in step b) (i) more than 20 equivalents of DIBAL-H are employed.

This invention further provides the instant process, wherein one, two, three or four hydroxyl groups of the terpene trilactone cage skeleton are removed. This invention further provides the instant process, wherein one, two or three lactones of the terpene trilactone cage skeleton are reduced. This invention further provides the instant process, wherein the terpene trilactone cage skeleton is ginkgolide J. This invention further provides the instant process, wherein the hydroxyl bearing terpene trilactone cage skeleton is ginkgolide B and the removal of a hydroxyl group produces ginkgolide A. This invention further provides the instant process, wherein the hydroxyl bearing terpene trilactone cage skeleton is ginkgolide C and the removal of a hydroxyl group produces ginkgolide B, J, or M. This invention further provides the instant process, wherein the hydroxyl bearing terpene trilactone cage skeleton is ginkgolide C and the removal of a hydroxyl group produces ginkgolide J.

This invention further provides the instant process, wherein the lactone bearing terpene trilactone cage skeleton is ginkgolide A which is reduced in step a) to:

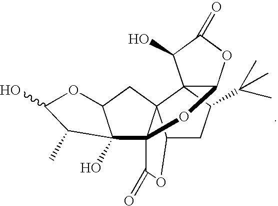

This invention further provides the instant process, wherein the hydroxyl bearing terpene trilactone cage skeleton is reduced in step b) (ii) to:

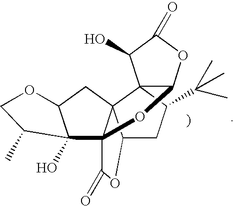

This invention further provides the instant process, wherein the hydroxyl bearing terpene trilactone cage skeleton is reduced to form a first product having the structure:

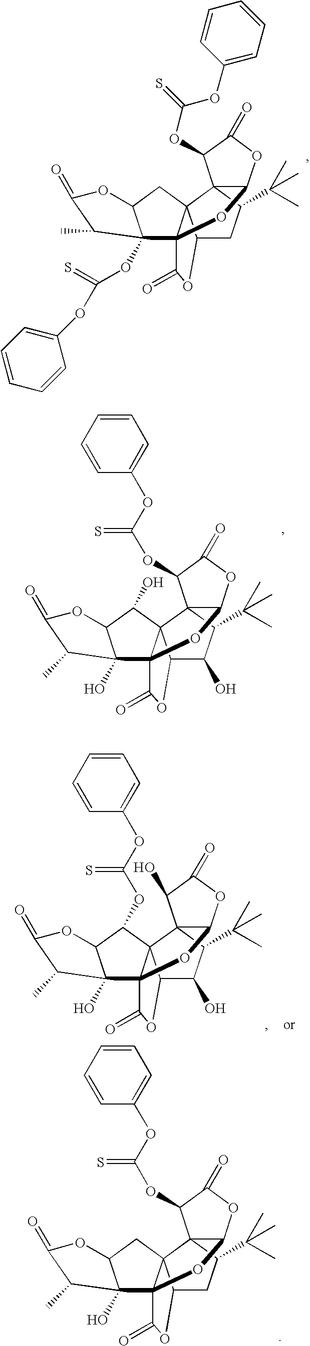

This invention further provides the instant process, wherein the hydroxyl group of the hydroxyl bearing terpene trilaxctone cage skeleton is removed to produce a compound having the following structure:

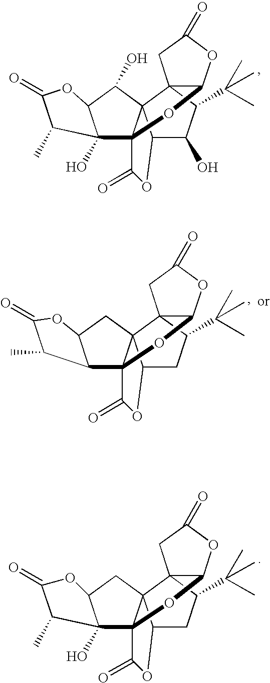

This invention further provides the instant process, wherein step a), step b), or step a) and step b), are performed more than once on a single lactone bearing and/or hydroxyl bearing terpene trilactone cage skeleton.

This invention further provides the instant process, wherein the lactone bearing terpene trilactone cage skeleton is ginkgolide A and the ginkgolide A is reduced to:

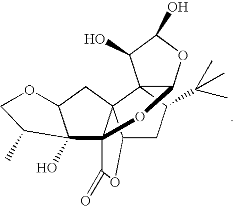

This invention further provides the instant process, wherein the lactone bearing terpene trilactone cage skeleton is ginkgolide A and the ginkgolide A is reduced to:

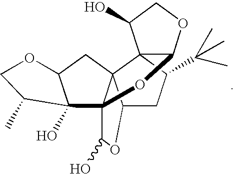

This invention further provides the instant process, wherein the terpene trilactone cage skeleton is ginkgolide A and the ginkgolide A is reduced to:

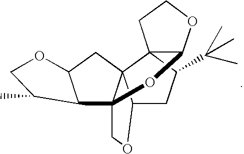

This invention further provides the instant process, wherein the terpene trilactone cage skeleton is ginkgolide A and the ginkgolide A is reduced to:

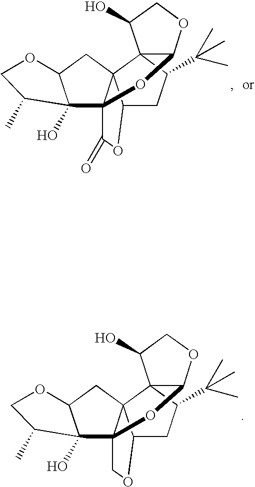

This invention further provides the instant process, wherein the terpene trilactone cage skeleton is reduced and/or has hydroxyl group(s) removed to produce a compound having one of the following structures:

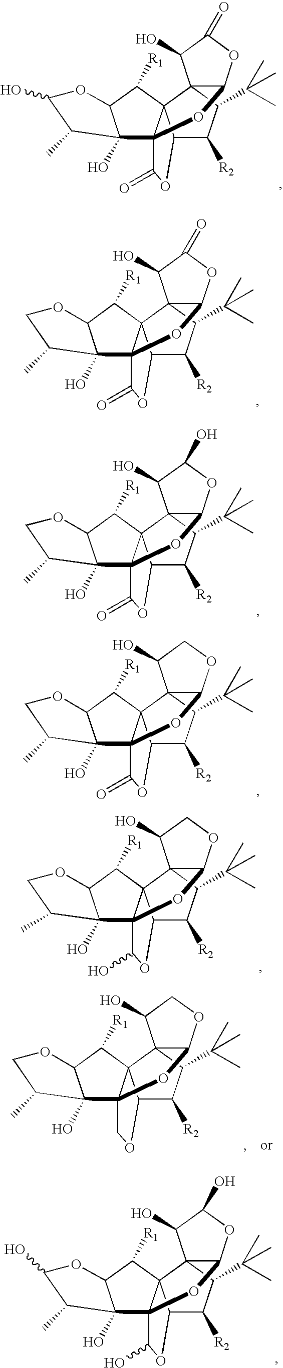

wherein R¹ and R² are, independently, H or OH.

This invention further provides the instant process, wherein the process produces a compound having one of the following structures:

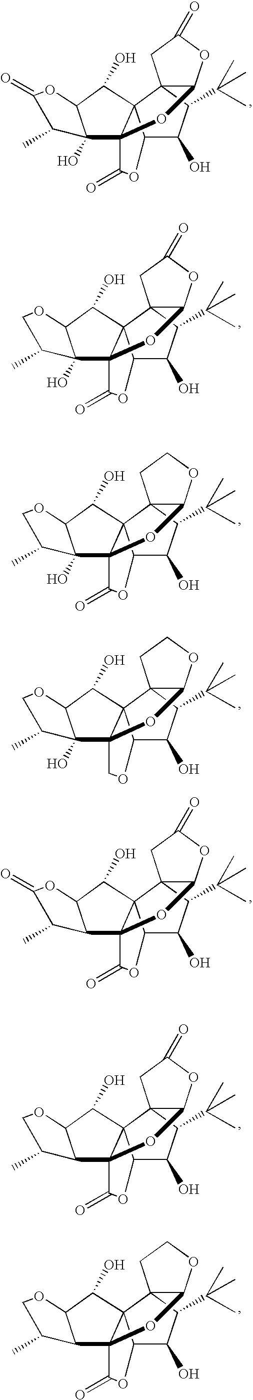

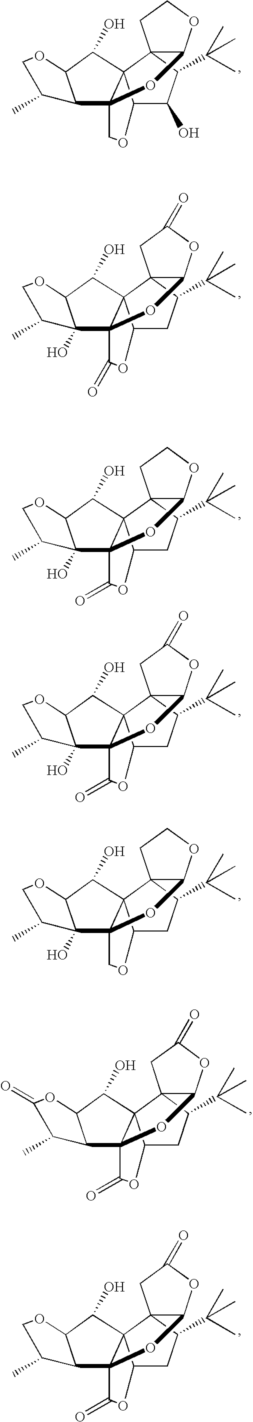

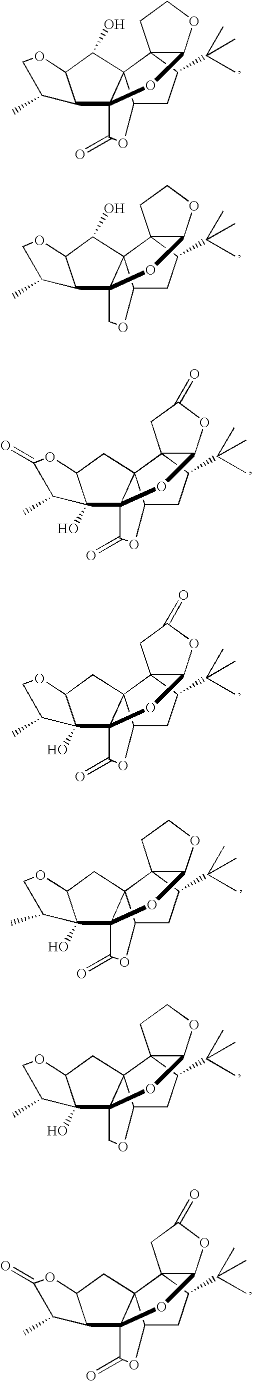

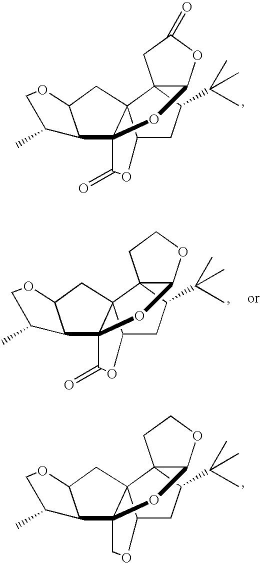

This invention further provides the instant process, wherein the process produces a compound having one of the following structures:

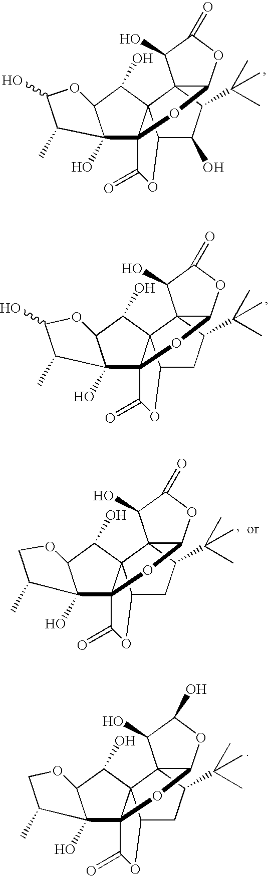

This invention further provides the instant process, wherein the process produces a compound having one of the following structures:

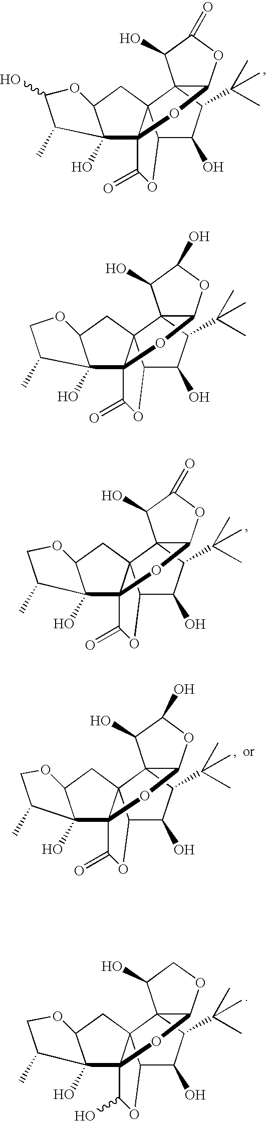

This invention provides the instant process for removing the hydroxyl group on the hydroxyl-bearing terpene trilactone cage skeleton or bilobalide, wherein in the process the hydroxyl group is removed by exposing the hydroxyl-bearing terpene trilactone cage skeleton or bilobalide to (diethylamino)sulfur trifluoride and pyridine in the presence of the sixth suitable solvent for a time sufficient to remove the hydroxyl group.

In one embodiment the hydroxyl group removed is a tertiary hydroxyl group. In one embodiment the sixth suitable solvent is THF.

In one embodiment the terpene trilactone is a ginkgolide. In further embodiments the ginkgolide is ginkgolide A, ginkgolide B, ginkgolide C or ginkgolide J. In one embodiment the terpene trilactone is a 10-benzyl-ginkgolide or a 10-methyl-ginkgolide. In further embodiments the ginkgolide is 10-benzyl-ginkgolide A, 10-benzyl-ginkgolide B, 10-benzyl-ginkgolide C, 10-benzyl-ginkgolide J or 10-benzyl-ginkgolide M, 10-methyl-ginkgolide A, 10-methyl-ginkgolide B, 10-methyl-ginkgolide C, 10-methyl-ginkgolide J or 10-methyl-ginkgolide M.

In one embodiment the terpene trilactone is a 10-benzyl-ginkgolide or a 10-methyl-ginkgolide and has the structure:

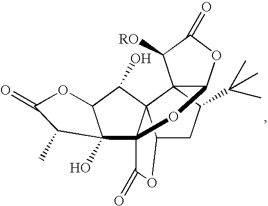

wherein R is Bn or Me and R₁ is H or OH.

This invention also provides the instant process for replacing the hydroxyl group on the hydroxyl-bearing terpene trilactone cage skeleton or bilobalide, wherein in the process the hydroxyl group is replaced by exposing the hydroxyl bearing terpene trilactone cage skeleton or bilobalide to an allylating agent and TiCl₄ or BF₃-Et₂0 in the presence of the seventh suitable solvent for a time sufficient to replace the hydroxyl group.

In one embodiment the hydroxyl group is replaced by an allyl functionality. In one embodiment the allylating agent has the structure:

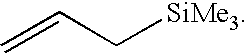

In one embodiment the seventh suitable solvent is CH₂Cl₂.

In one embodiment the hydroxyl group of the terpene trilactone cage skeleton is obtained by exposing a lactone bearing terpene trilactone cage skeleton or bilobalide to DIBAL-H in an eighth suitable solvent to form a resulting terpene trilactone cage skeleton having a hydroxyl group at the position of the lactone. In one embodiment the eighth suitable solvent is CH₂Cl₂.

This invention provides the instant process wherein the hydroxyl group is replaced by an allyl functionality and produces a compound having the structure:

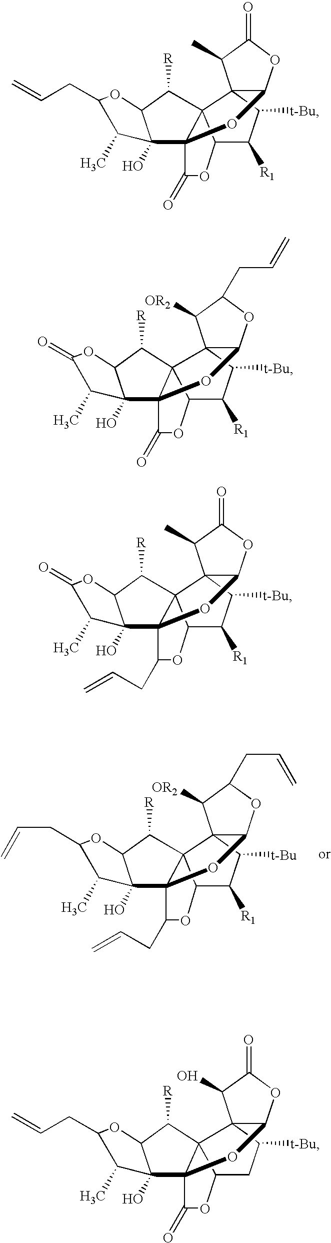

wherein R, R₁ and R₂ are, independently, H, OH, an alkyl, an aryl or a functional group.

This invention also provides a process of increasing the hydrophobicity of a lactone bearing terpene trilactone cage skeleton comprising reducing one or more lactones of the lactone bearing terpene trilactone by exposing it to DIBAL-H.

This invention also provides a process for making ginkgolide J from ginkgolide C comprising:
a) exposing the ginkgolide C to a compound having the following structure:

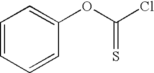

in the presence of DMAP and a suitable solvent so as to make a product having the structure:

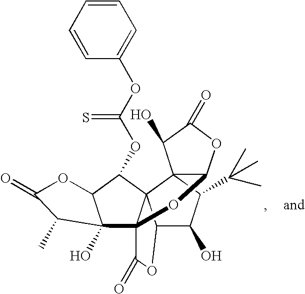

b) exposing the product of step (a) to Et₃SiH and Bz₂0 or Bu₃SnH and AlBN, in the presence of a suitable solvent, and refluxing to produce ginkgolide J.

This invention also provides the instant process, wherein the suitable solvent in step a) is CH₃CN. This invention also provides the instant process, wherein the suitable solvent in step b) is toluene.

This invention also provides a process for making a ginkgolide triether from a ginkgolide A or ginkgolide J comprising:
a) exposing the ginkgolide to a suitable reducing agent in a suitable solvent so as to so as to reduce lactones of the terpene trilactone to lactols; and
b) exposing the product of step a) to Et₃SiH and BF₃-Et₂0 in a suitable solvent for sufficient time to deoxygenate the lactols to cyclic ethers so as to thereby make the ginkgolide triether.

This invention also provides the instant process, wherein step a) is performed at −70° C. to −80° C. This invention also provides the instant process, wherein step b) is performed at −45° C. to −55° C.

This invention also provides the instant process, wherein the ginkgolide is ginkgolide A and the product of step a) has the structure:

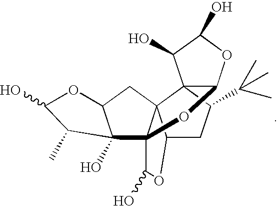

This invention also provides the instant process, wherein the ginkgolide triether has the structure:

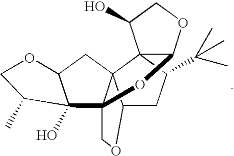

This invention further provides the instant process, wherein the ginkgolide triether has the structure:

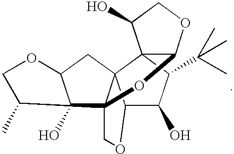

This invention further provides the instant process, wherein the suitable solvent in step a) is THF. This invention also provides the instant process, wherein the suitable solvent in step b) is dichloromethane. This invention also provides the instant process, wherein the suitable reducing agent is DIBAL-H.

This invention further provides process of producing ginkgolide M comprising:
(a) exposing 10-benzyl-ginkgolide C or 10-methyl-ginkgolide C to pyridine and (diethylamino)sulfur trifluoride in the presence of a suitable solvent so as to produce a compound having the structure:

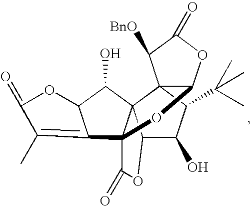

(b) exposing the product of step (a) to H₂ under pressure in the presence of Pd/C so as to produce 14-epi-ginkgolide M having the structure:

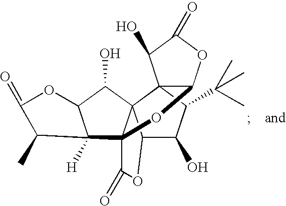

(c) exposing the 14-epi-ginkgolide M of step (b) to DMAP in a suitable solvent for a time sufficient to produce ginkgolide M.

In an embodiment the H₂ is under 4-6 atmospheres of pressure. In a further embodiment the H₂ is under about 5 atmospheres of pressure. In an embodiment the suitable solvent in step (a) is THF. In a further embodiment the suitable solvent in step (c) is CH₃CN.

This invention further provides a process for producing a 10-substituted ginkgolide derivative comprising exposing a ginkgolide having a hydroxyl group at the 10-position to a compound having the structure:

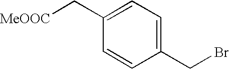

in the presence of a suitable base and a suitable solvent for a time sufficient to produce the 10-substituted ginkgolide derivative. In an embodiment the suitable solvent is DMF, THF or CH₃CN. In one embodiment the suitable solvent is DMF. In an embodiment the suitable base is NaH, KH, Na₂CO₃, K₂CO₃ or iPr₂EtN. In one embodiment the suitable base is K₂CO₃.

This invention provides the instant process wherein the ginkgolide is ginkgolide B and the 10-substituted ginkgolide derivative has the structure:

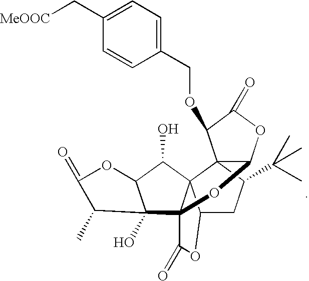

In an embodiment the ginkgolide is ginkgolide C, ginkgolide J or ginkgolide A.

This invention also provides a process for producing a 10-substituted ginkgolide derivative comprising exposing a ginkgolide having a hydroxyl group at the 10-position to MeI in the presence of a suitable base and a suitable solvent for a time sufficient to produce the 10-substituted ginkgolide derivative. In an embodiment the suitable solvent is DMF, THF or acetone. In one embodiment the suitable solvent is acetone. In an embodiment the suitable base is NaH, KH or K₂CO₃. In one embodiment the suitable base is K₂CO₃.

This invention provides the instant process wherein the ginkgolide having the hydroxyl group at the 10-position is ginkgolide C and the 10-substituted ginkgolide derivative has the structure:

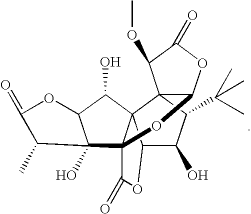

In one embodiment of the instant process the ginkgolide is ginkgolide B, ginkgolide A or ginkgolide J.

In an embodiment of the instant process the ginkgolide is ginkgolide A and the suitable base is KH.

This invention also provides a process for producing a 7-substituted ginkgolide derivative comprising exposing a ginkgolide having a hydroxyl group at the 7-position to a compound having the structure:

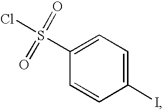

in the presence of a suitable base and a suitable solvent for a time sufficient to produce the 7-substituted ginkgolide derivative. In an embodiment the suitable solvent is CH₂Cl₂ or CHCl₃. In one embodiment the suitable solvent is CH₂Cl₂. In an embodiment the suitable base is iPr₂EtN, DMAP, Et₃N or pyridine. In an embodiment the suitable base is iPr₂EtN. In an embodiment the ginkgolide is ginkgolide C and the 7-substituted ginkgolide derivative has the structure:

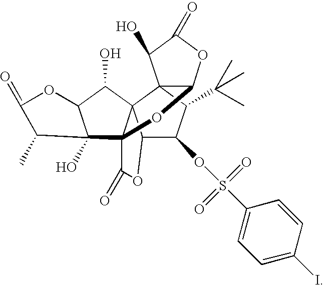

This invention also provides a compound having the following structure:

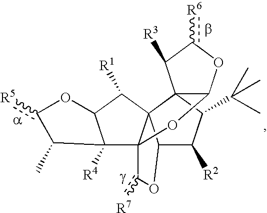

wherein each of R¹, R², and R⁴ is, independently, H or OH; each of R⁵, R⁶ and R⁷ is H or OH, or O and the respective bond α, β, or γ is present; and
R³ is H, or
R³ is OH when R¹ is H, R² is OH and R⁴ is H,
   or when at least one of R⁵, R⁶ and R⁷ is OH,
   or when R⁵ is H, R⁶ is O and bond β is present and R⁷ is H,
wherein R⁵ is H or OH when only one of R⁶ or R⁷ is O.

This invention further provides the instant compound, wherein
each of R¹, R², and R⁴ is, independently, H or OH;
each of R⁵, R⁶ and R⁷ is O and the respective bond α, β, or γ is present; and
R³ is H, or
R³ is OH when R¹ is H, R² is OH and R⁴ is H.

This invention further provides the instant compound, wherein
each of R¹, R², and R⁴ is, independently, H or OH;
at least one of R⁵, R⁶ and R⁷ is H or OH; and
R³ is H, or
R³ is OH when at least one of R⁵, R⁶ and R⁷ is OH.

This invention further provides the instant compound, wherein at least two of R⁵, R⁶ and R⁷ are H or OH.

This invention further provides the instant compound, wherein
each of R¹, R², and R⁴ is, independently, H or OH;
at least one of R⁵, R⁶ and R⁷ is H or OH; and
R³ is H, or
R³ is OH when R⁵ is H, R⁶ is O and bond β is present and R⁷ is H.

This invention further provides the instant compound, wherein at least two of R⁵, R⁶ and R⁷ are H or OH.

This invention further provides the instant compound, having the structure:

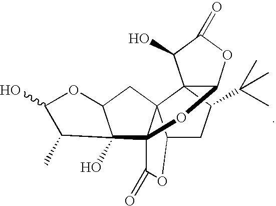

This invention further provides the instant compound, having the structure:

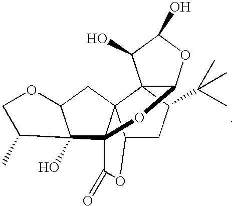

This invention further provides the instant compound, having the structure:

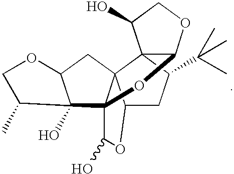

This invention further provides the instant compound, compound having the structure:

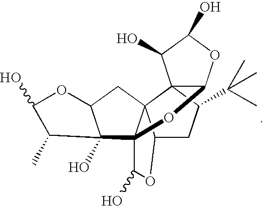

This invention further provides the instant compound, having the structure:

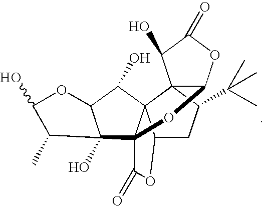

This invention further provides the instant compound, the structure:

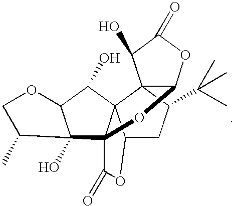

This invention further provides the instant compound, having the structure:

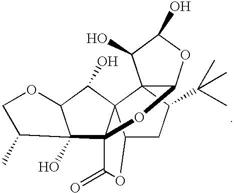

This invention further provides the instant compound, having the structure:

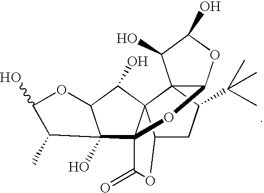

This invention further provides the instant compound, having one of the following structures:

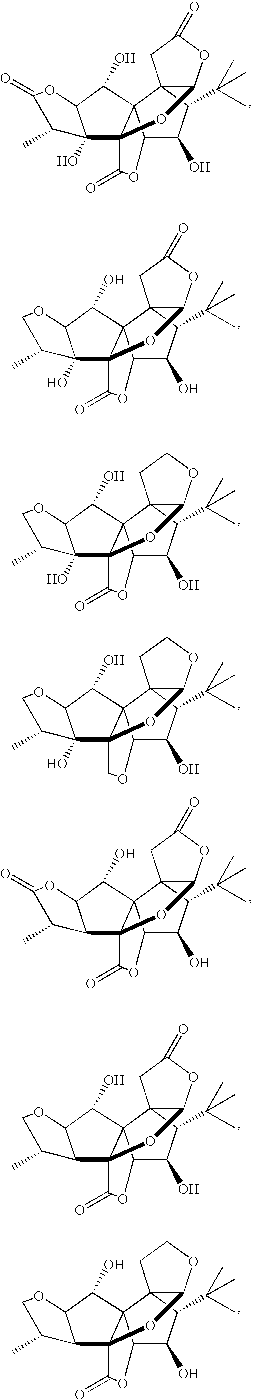

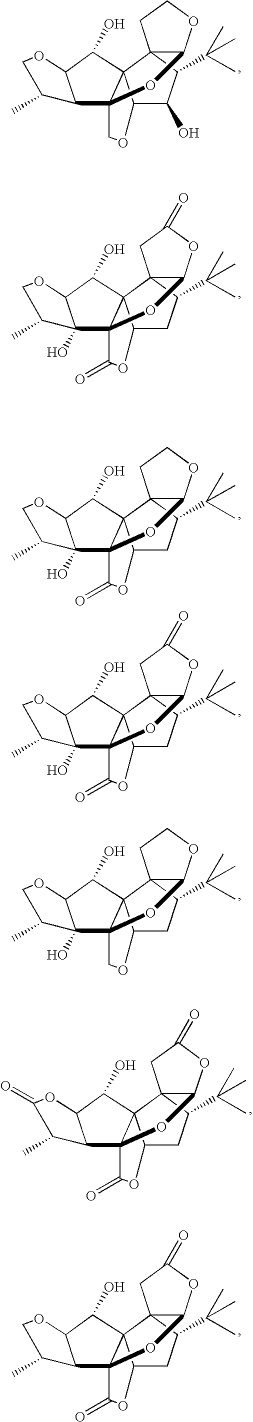

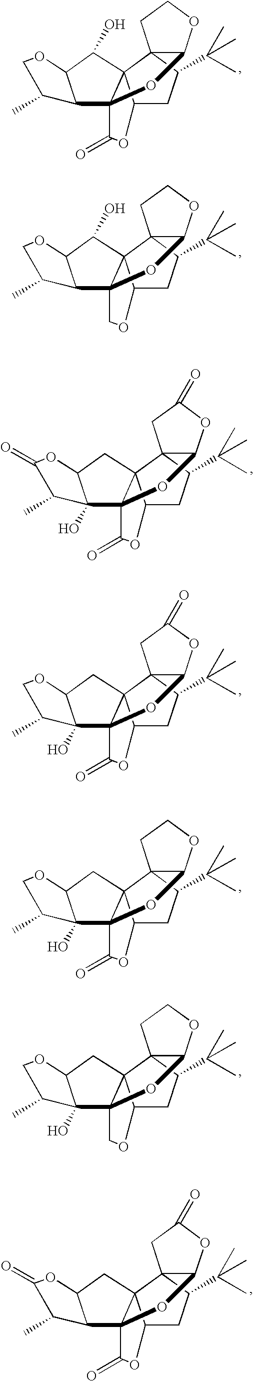

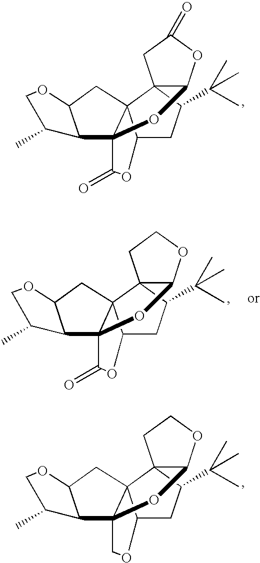

This invention further provides the instant compound, having one of the following structures:

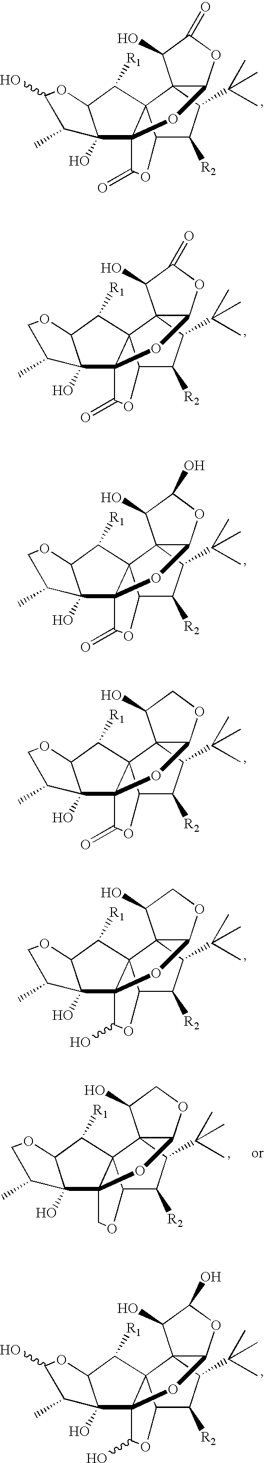

wherein R¹ and R² are independently, H or OH.

This invention further provides the instant compound, having the following structure:

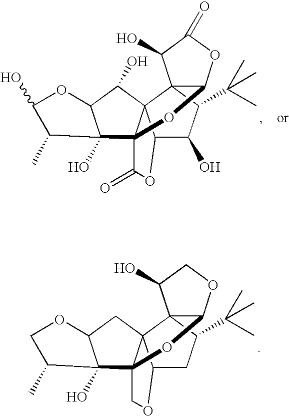

This invention further provides the instant compound, wherein the compound has one of the following structures:

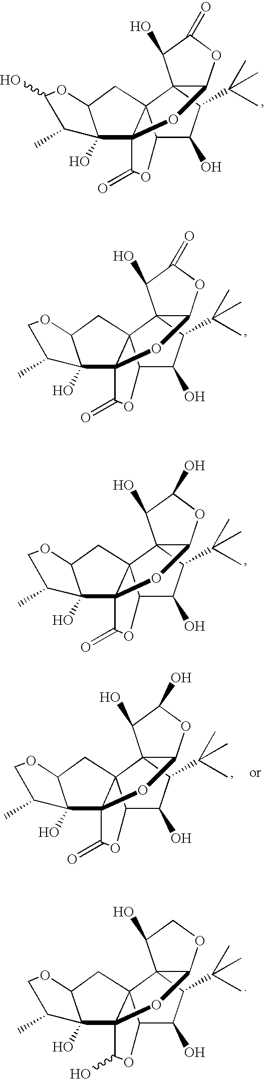

This invention further provides the instant compound, having the following structure:

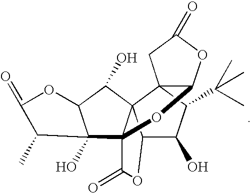

This invention also provides a compound, having the structure:

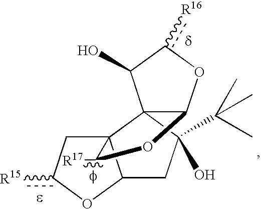

wherein one of R¹⁵, R¹⁶, or R¹⁷ is H or OH, and wherein when R¹⁵, R¹⁶, or R¹⁷ is O, the respective bond δ, ε, or φ is present.

This invention also provides a method of determining whether a test compound is a platelet-activating factor (PAF) receptor antagonist or agonist comprising:
a) quantitating the activity of a platelet-activating factor PAF receptor in a PAF receptor-containing membrane or tissue in the presence of a predetermined amount of a PAF receptor agonist;
b) exposing the PAF receptor to a predetermined amount of any one of the instant compounds;
c) quantitating the reduction of the PAF receptor activity in the presence of both the predetermined amount of PAF receptor agonist and the predetermined amount of any one of the instant compounds; and
d) exposing the PAF receptor to the test compound and quantitating the reduction or increase of the PAF receptor activity in the presence of the test compound as compared to the PAF receptor activity quantitated in step c),
   whereby an increase in PAF receptor activity quantitated in step d) as compared to step c) indicates that the test compound is a PAF receptor agonist, and whereby a decrease in PAF receptor activity quantitated in step d) as compared to step c) indicates that the test compound is a PAF receptor antagonist.

This invention also provides a method of determining whether a test compound relieves or enhances impairment of long-term potentiation (LTP) by a beta amyloid comprising:
a) quantifying a LTP in a mammalian brain portion;
b) exposing the brain to a predetermined amount of the beta amyloid and quantifying the impairment of the LTP in the mammalian brain portion in the presence of the beta amyloid;
c) exposing the brain to a predetermined amount of a compound of any one of the instant compounds sufficient to reduce the impairment of the LTP in the mammalian brain portion by the beta amyloid; and
d) exposing the brain to the test compound and quantitating the reduction or increase of the LTP in the mammalian brain portion in the presence of the test compound as compared to the LTP quantitated in step c),
   whereby an increase in LTP quantitated in step d) as compared to step c) indicates that the test compound relieves impairment of LTP by beta amyloid, and whereby a decrease in LTP quantitated in step d) as compared to step c) indicates that the test compound enhances beta-amyloid impairment of LTP.

This invention further provides the instant method, wherein the mammalian brain portion is a hippocampal slice. This invention further provides the instant method, wherein the LTP is measured in the CA1 region of the hippocampal slice. This invention further provides the instant method, wherein the beta amyloid is Aβ₁₋₄₂.

This invention also provides a method of determining whether a test compound inhibits neuronal cell death comprising:
a) exposing a first plurality of neuronal cells to a compound of any one of the instant compounds;
b) exposing the first plurality of neuronal cells to a predetermined amount of beta amyloid;
c) determining the rate of neuronal cell death of the first plurality of neuronal cells at a predetermined time after steps a) and b)
d) exposing a second plurality of the neuronal cells to the test compound;
e) exposing the second plurality of the neuronal cells to the predetermined amount of beta amyloid;
f) determining the rate of neuronal cell death of the second plurality of the neuronal cells at a predetermined time after steps d) and e); and
g) comparing the rate of neuronal cell death determined in step f) to that determined in step c),
   whereby a lower rate of neuronal cell death determined in step f) as compared to step c) indicates that the test compound inhibits neuronal cell death.

This method also provides a process for methylating a C10 hydroxyl and/or a C3 hydroxyl of hydroxyl bearing terpene trilactone cage skeleton comprising exposing the terpene trilactone cage skeleton to MeI and KH in a suitable solvent for a sufficient time to methylate the C10 hydroxyl and/or the C3 hydroxyl of the terpene trilactone cage skeleton. This invention further provides the instant process, wherein 50 Eq of MeI are used. This invention further provides the instant process, wherein the suitable solvent is THF. This invention further provides the instant process, wherein the process is performed at or about room temperature. This invention further provides the instant process, wherein the hydroxyl bearing terpene trilactone cage skeleton is ginkgolide A and the process produces a compound having the structure:

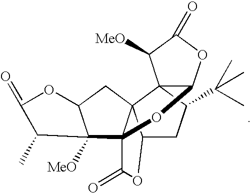

This invention also provides a process for methylating a C10 hydroxyl and a C3 hydroxyl of a ginkgolide triether comprising exposing the ginkgolide triether to MeI, AgOTf, and Et₃N in a suitable solvent and refluxing to methylate the C10 hydroxyl and the C3 hydroxyl of the ginkgolide triether. This invention further provides the instant process wherein 10 Eq of MeI are used. This invention further provides the instant process, wherein the suitable solvent is THF. This invention further provides the instant process, wherein the ginkgolide triether is ginkgolide A triether and the process produces a compound having the structure:

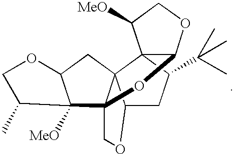

This invention also provides a compound having the following structure:

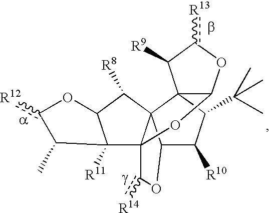

wherein each of R⁸, R⁹ and R³¹ are, independently, H, OH, OMe or

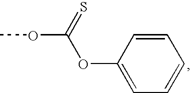

with the proviso that at least two of R⁸, R⁹ and R¹¹ are Ome or at least one of R⁸, R⁹ and R¹¹ is

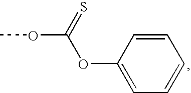

and each of R¹², R¹³ and R¹⁴ is H or OH, or O and the respective bond α, β, or γ is present, and R¹⁰ is H or OH.

This invention further provides the instant compound, wherein the compound has one of the following structures:

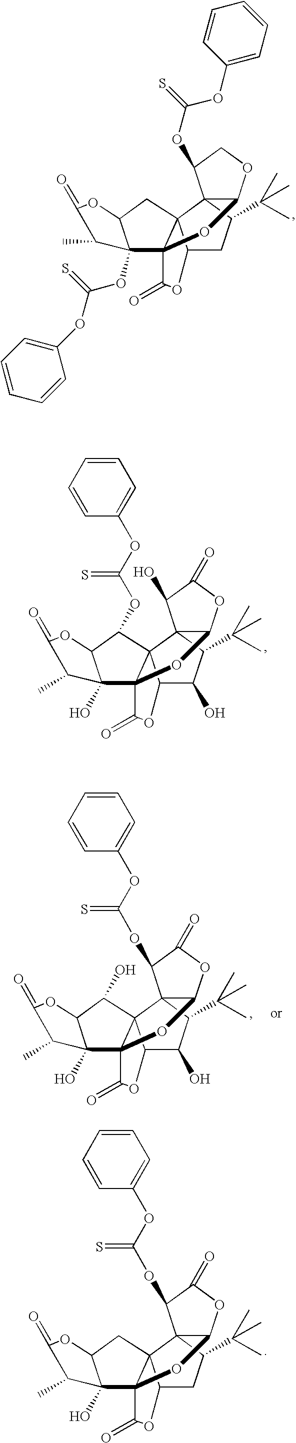

This invention further provides the instant compound, wherein the compound has one of the following structures:

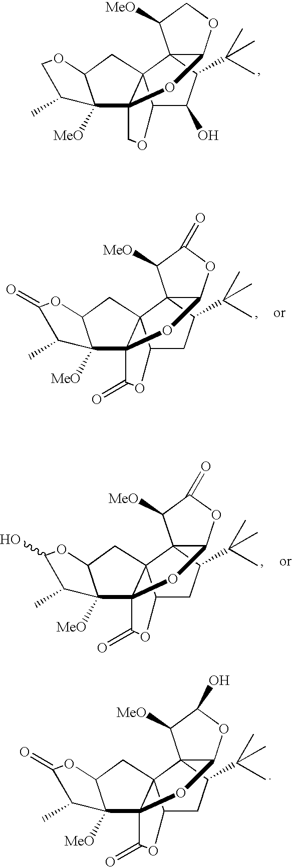

This invention also provides a compound having one of the following structures:

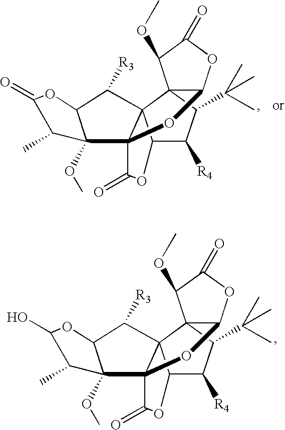

wherein R₃ and R₄ are, independently, H or OMe. These compounds may be made by the methylation processes described hereinabove.

This invention provides a process of functionalizing a terpene trilactone cage skeleton at a C1, C7, or C10 position comprising exposing the terpene trilactone cage skeleton to an alkylating agent capable of undergoing a subsequent deoxygenation, in the presence of DMAP and a second suitable solvent to form a first product. This invention further provides the instant process, wherein the alkylating agent is PhOC(S)Cl, the suitable solvent is DMF and the terpene trilactone cage skeleton is functionalized with PhOC(S) at the at a C1 position. This invention further provides the instant process, wherein the alkylating agent is PhOC(S)Cl, the suitable solvent is THF or CH₃CN, and the terpene trilactone cage skeleton is functionalized with PhOC(S) at the at a C10 position. This invention further provides the instant process, wherein the alkylating agent is PhOC(S)Cl, the terpene trilactone cage skeleton has previously been functionalized at the C1 or C10 position and the process functionalizes the terpene trilactone cage skeleton at the C10 position. This invention further provides the instant process, wherein the alkylating agent is R—Cl and the process functionalizes the terpene trilactone cage skeleton with R at the C7 or C10 position. This invention further provides the instant process, further comprising increasing the amount of alkylating agent present so as functionalize two or more of C1, C7, and C10 simultaneously.

This invention provides a process of removing a hydroxyl group on a terpene trilactone cage skeleton or a bilobalide comprising exposing a hydroxyl bearing terpene trilactone cage skeleton or bilobalide to an alkylating agent capable of undergoing a subsequent deoxygenation, in the presence of a base and a first suitable solvent to form a first product, and exposing the first product to Bu₃SnH and AlBN in the presence of a second suitable solvent for a time sufficient to deoxygenate the hydroxyl group, so as to thereby remove the hydroxyl group from the terpene trilactone cage skeleton or bilobalide. In one embodiment the terpene trilactone cage skeleton is ginkgolide C. In embodiments the first suitable solvent is DMF or CH₃CN, the second suitable solvent is toluene/EtOH, the alkylating agent is PhOC(S)Cl.

This invention provides the instant process wherein the terpene trilactone cage skeleton is ginkgolide C, the alkylating agent is PhOC(S)Cl, the base is DMAP, the first suitable solvent is DMF, the second suitable solvent is toluene/EtOH and the C1 hydroxyl group is removed, or wherein the terpene trilactone cage skeleton is ginkgolide C, the alkylating agent is PhOC(S)Cl, the base is DMAP, the first suitable solvent is CH₃CN, the second suitable solvent is toluene/EtOH and the C10 hydroxyl group is removed.

This invention also provides the instant process producing a compound having the structure:

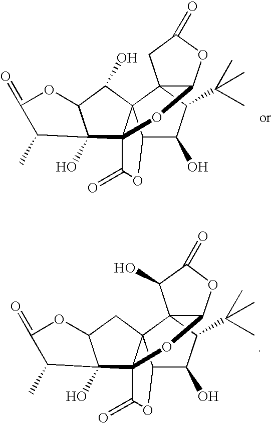

and/or producing a first product having the structure:

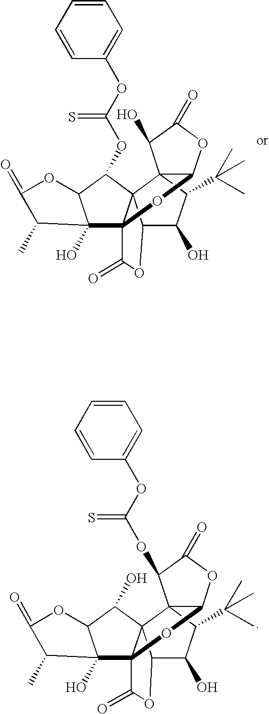

This invention provides the instant process wherein the base is pyridine, N-methylimidazole or Et₃N, and/or wherein the first suitable solvent is dioxane, EtOAc, THF, N,N-dimethylacetamide or pyridine.

This invention provides a process of producing ginkgolide J comprising exposing ginkgolide C to an alkylating agent capable of undergoing a subsequent deoxygenation, in the presence of a base and a first suitable solvent to form a first product, and exposing the first product to Bu₃SnH and AlBN in the presence of a second suitable solvent for a time sufficient to deoxygenate a C1 hydroxyl group of the ginkgolide C, so as to thereby produce ginkgolide J. In one embodiment the alkylating agent is PhOC(S)Cl, the base is DMAP, the first suitable solvent is DMF, and the second suitable solvent is toluene/EtOH. In embodiments of this process the base is DMAP and in excess of 1 equivalent of DMAP is used, or the base is DMAP and in excess of 2 equivalents of DMAP is used.

This invention also provides a compound having the structure:

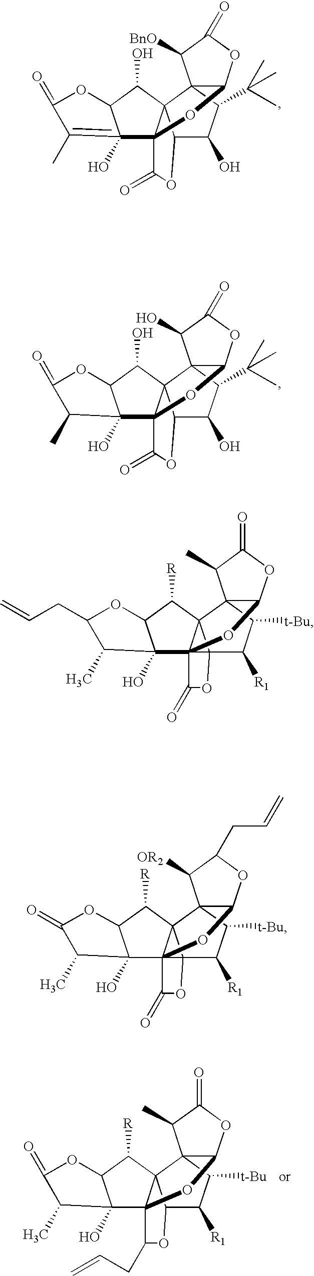

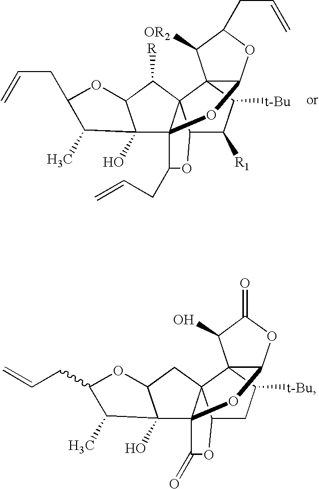

wherein R, R₁ and R₂ are, independently, H, OH, an alkyl, an aryl or a functional group.

This invention also provides a process for double dehydrating a ginkgolide comprising exposing the ginkgolide to pyridine and SOCl₂. In one embodiment the ginkgolide is ginkgolide C and the double dehydrated product has the structure:

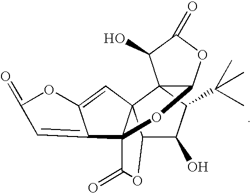

This invention also provides a compound having the structure:

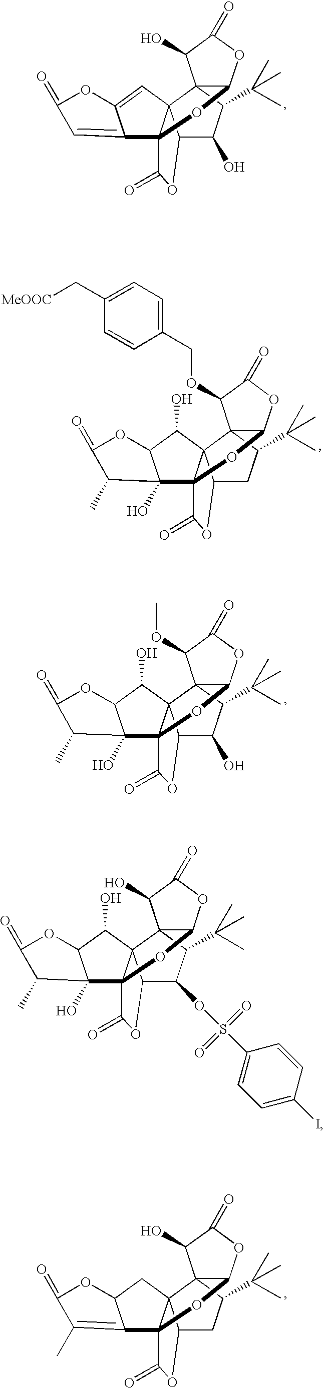

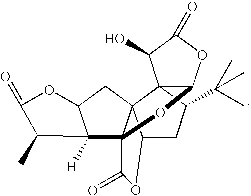

This invention also provides a process for making ginkgolide L from ginkgolide A comprising exposing the ginkgolide A to (diethylamino)sulfur trifluoride in the presence of a suitable solvent for a time sufficient to produce ginkgolide L. In one embodiment the ginkgolide L so produced has the structure:

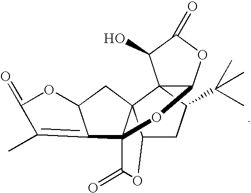

This invention also provides a process of making a compound having the structure:

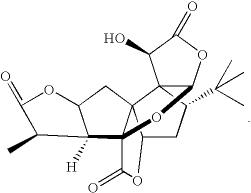

comprising exposing a compound having the structure

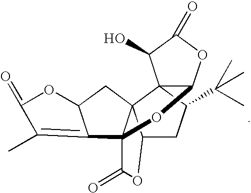

to H₂ under pressure in the presence of Pd/C so as to produce the compound. In one embodiment the H₂ is under 4-6 atmospheres of pressure. In a further embodiment the H₂ is under about 5 atmospheres of pressure.

A “terpene trilactone” as used herein refers to the ginkgolides GA, GB, GC, GJ, and GM as well as bilobalide.

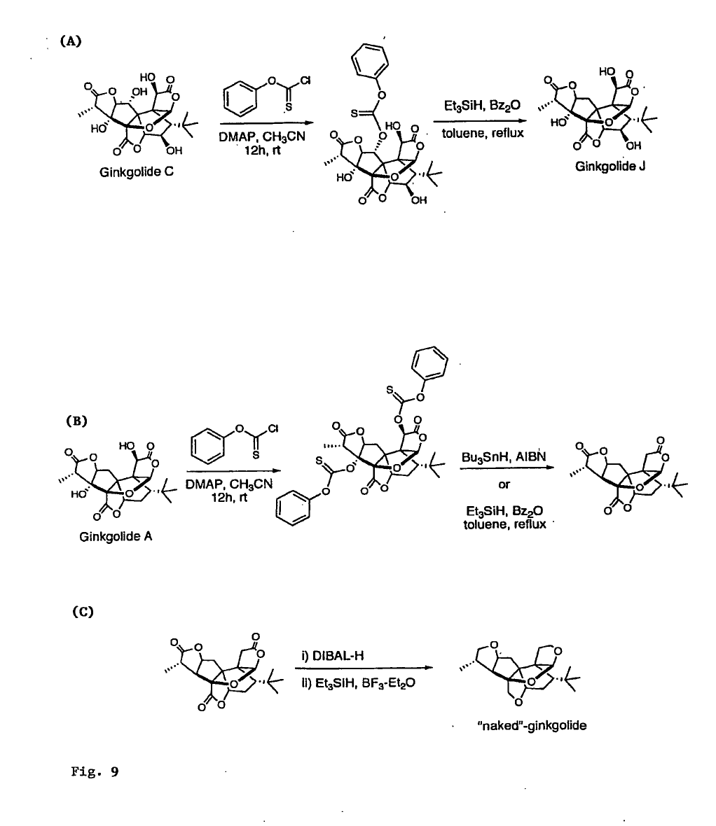

A “terpene trilactone cage skeleton” refers to the joined six 5-membered rings that constitute the common core between the naturally occurring ginkgolide A, B, C, J and M. “Terpene trilactone cage skeleton” as used herein, however, refers to the structure regardless of whether it is part of a molecule obtained from a natural source or synthetically made. The terpene trilactone cage skeleton which does not bear any lactone, lactol or hydroxyl group is referred to herein as a “naked” ginkgolide whose structure is shown in FIG. 9(c).

Any of the first, second, third, fourth, fifth, sixth, seventh and eighth suitable solvents referred to herein may be different from the remaining solvents, but may also be the same as one or more of the first to eighth solvents. For example, the first solvent may be the same as the second, third, fourth and fifth solvents and so forth, or the first solvent may be the same as the second and third solvents, but different than the fourth and fifth solvents, or the first solvent may be dissimilar to any of the second to fifth solvents. Each of the solvents is independently chosen based on its suitability for the reaction being performed. First suitable solvents include THF, THF/Hexane, dichloromethane (CH₂Cl₂) and dioxane. Second suitable solvents include CH₃CN, DMF, THF, dioxane, and dichlromethane (CH₂Cl₂). Third and fourth suitable solvents include toluene, CH₂Cl₂, benzene, chloroform and THF. Fifth suitable solvents include THF, dichloromethane (CH₂Cl₂), and dioxane. Sixth suitable solvents include THF. Seventh suitable solvents include CH₂Cl₂. Eighth suitable solvents include CH₂Cl₂. These examples are non-limiting.

The methods disclosed here for removing hydroxyl groups or lactones may be applied to bilobalide also.

As used in the structural diagrams herein, a wavy line bond denotes a bond that has variable 3-D geometry, i.e. either comes out of, or goes into, the plane of the paper.

As used herein, “room temperature” means between 18° C. and 27° C., and more preferably 20-25° C.

The compounds of this invention may be used in formulations or compositions to treat neurodegenerative disorders including, but without limitation, Alzheimer's disease and variants thereof, dementias, as well as PAF-receptor associated diseases. The compounds may be administered directly or in the form of salts, and as part of compositions which may comprise pharmaceutically acceptable components.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. For example, an amount effective to inhibit or reverse a neurodegenerative disorder or attenuate or reverse the disorder symptoms. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

As used herein, a “salt” is salt of the instant compounds which has been modified by making acid or base salts of the compounds. In the case of compounds used for treatments, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium.

As used herein, a “pharmaceutically acceptable carrier” is a pharmaceutically acceptable solvent, suspending agent or vehicle, for delivering the instant compounds to an animal or human. The carrier may be liquid or solid and is selected with the planned manner of administration in mind. Liposomes are also a pharmaceutical carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds may comprise a single compound or mixtures thereof with other compounds. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection or other methods, all using dosage forms well known to those of ordinary skill in the pharmaceutical arts.

The compounds can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Specific examples of pharmaceutical acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.).

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The active ingredient can be administered orally in solid dosage forms, such as capsules, tablets, and powders, or in liquid dosage forms, such as elixirs, syrups, and suspensions. It can also be administered parentally, in sterile liquid dosage forms.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The instant compounds may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

The present invention also includes pharmaceutical kits, which comprise one or more containers containing a pharmaceutical composition comprising an effective amount of one or more of the compounds. Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

All combinations of the various elements are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Lactol-Free Terpene Trilactones

In brief, lactone-rings of ginkgolides can be converted into the corresponding tetrahydrofuran moieties via DIBAL-H reduction followed by deoxygenation of the formed lactols with Et₃SiH/BF₃.Et₂O producing a series of core-modified derivatives.

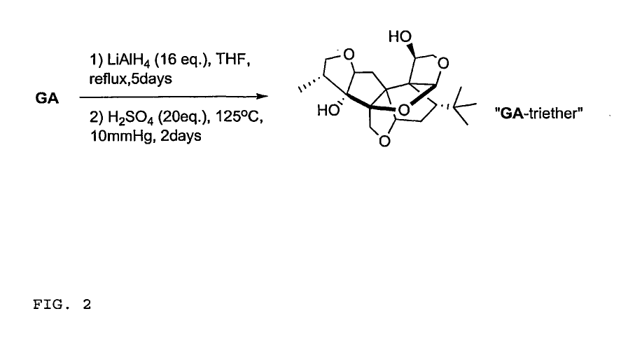

Initially, it was discovered that the previously reported synthesis of “GA-triether” did not give reproducible results (3a). Even a modified method (FIG. 2) suffered from low, inconsistent yields (5-20%), producing a variety of identified side products.

Furthermore, no partially reduced GA derivatives, such as those where lactone-C is reduced and lactone-E and -F stay intact, for example, could be detected. Furthermore, this approach failed to give any reduced derivatives of GB and GC. Also, the combinations of LiAlH₄ with Lewis acids, such as LiAlH₄/AlCl₃ (4) and LiAlH₄/BF₃.Et₂O (5) to reduce the lactone rings of GA and GC were unsuccessful.

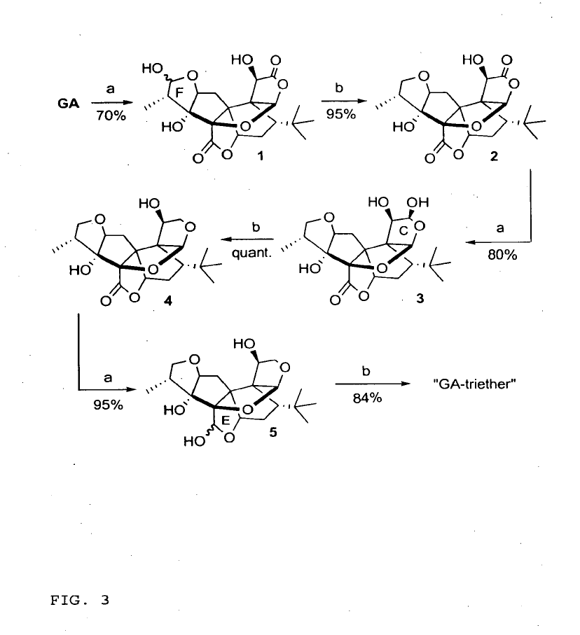

Therefore, we tested several other reducing agents (BH₃.Me₂S, L-selectride and DIBAL-H). Only the use of DIBAL-H (condition (a)) resulted in a clean reduction of GA into the corresponding lactol 1 (FIG. 3), as a 3:2 mixture of diastereomemers (determined by ¹H NMR), which have not been separated, and subjected to deoxygenation using Et₃SiH/BF₃.Et₂O protocol (6) (condition (b)) to yield diether 2. Reduction of 2 produced lactol 3 as a single (syn-“diol”) product. This indicated that the stereochemistry of 10-hydroxy groups controls the hydride attack. Reduction of 4 was done cleanly producing an approximately 1:1 ratio of diastereoisomers (determined by ¹H NMR), which were not separated and converted into “GA-triether” in high yield. Thus, this reduction/deoxygenation sequence created an entry point to a variety of novel core-modified hydrophobic ginkgolides.

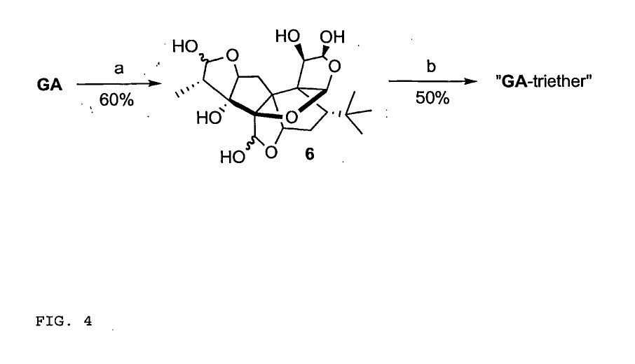

Next, this two-step protocol was applied for the direct conversion of GA into “GA-triether” (FIG. 4). Using a large excess amount of DIBAL-H afforded tri-lactol 6, which was isolated and converted into “GA-triether”. Unfortunately, direct transformation of GA into 4 turned out to be impractical, as an inseparable mixture of various lactols was observed.

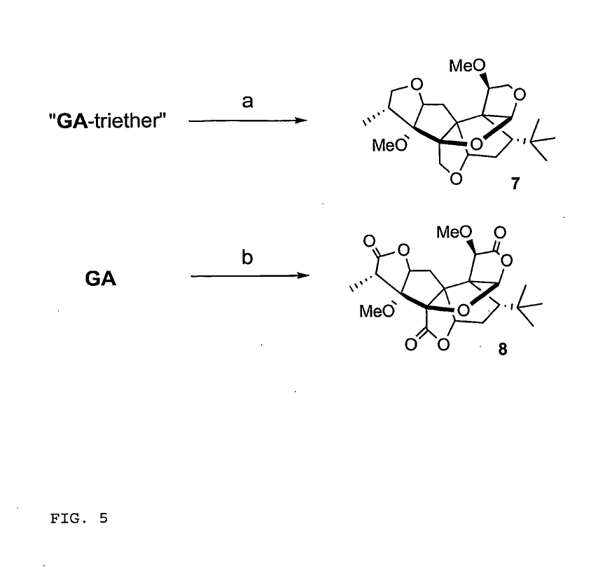

In addition, to further increase the hydrophobicity of the “GA-triether”, we performed methylation of the hydroxy-groups. “Per-etherated” ginkgolide derivative 7 was obtained in quantitative yield upon AgOTf-mediated methylation with MeI on “GA-triether” (FIG. 5).

Noteworthy is the fact that permethylation of GA under condition (a) as described in the figure description of FIG. 5, did not take place at all, and it required the use of KH to obtain dimethylated GA, 8, in ca. 40% yield. No methylation took place in the presence of NaH or K₂CO₃, resulting in a recovery of GA.

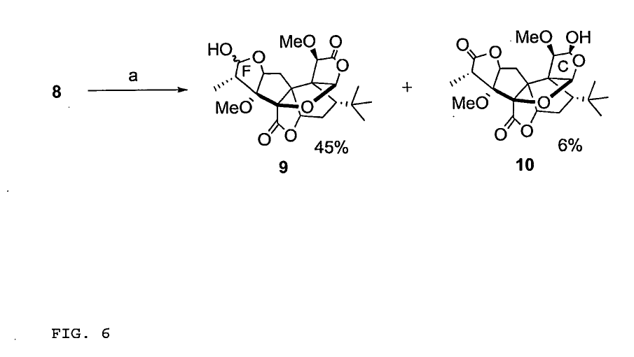

Reduction of 8 took place predominately at lactone-F, giving lactol 9 as a 7:3 mixture of diastereomers (determined by ¹H NMR), and giving lactol 10 as the minor product (FIG. 6, same conditions as FIG. 3).

Taken together with the reduction of GA, these results indicate that DIBAL-H reduction is sterically controlled, since the lactone-F is the least hindered.

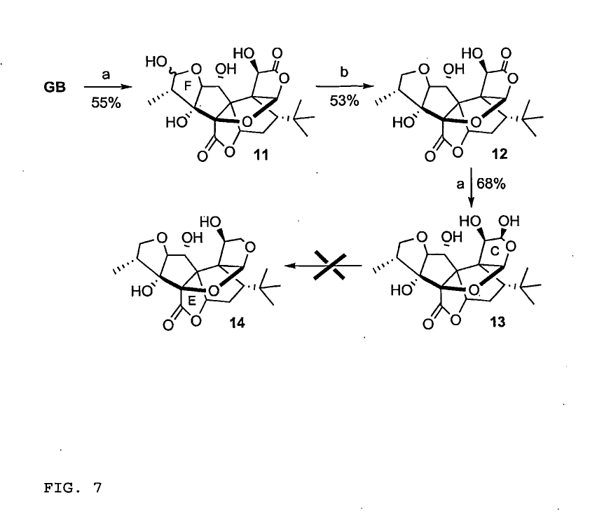

Similar to the reduction of GA, GB was reduced to the corresponding analogs 11 and 12 (FIG. 7). However, subsequent transformation of lactol 13 to 14 failed.

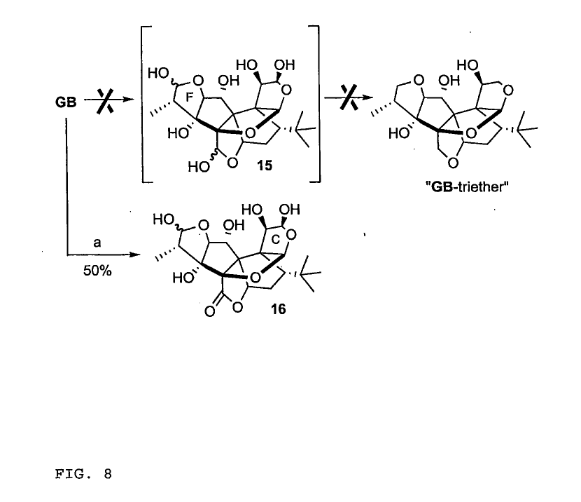

Additionally, in line with FIG. 4, we attempted the direct conversion of GB in to corresponding “GB-triether” via trilactol 16 (FIG. 8). Unfortunately, even under stringent conditions using large excess DIBAL-H (>25 eq.) dilactol 17 was the only reduced product detected; the subsequent deoxygenation did not take place.

The deoxygenation of GC turned out to be problematic as well. Only the reduction of lactone-F was achieved, which failed to undergo the subsequent conversion into the corresponding ether. The use of excess of DIBAL-H, 25 eq., gave an inseparable mixture of the dilactol and trilactol.

In conclusion, we have demonstrated that GA lactone rings can be reduced to the tetrahydrofuran moieties via regioselective DIBAL-H reduction—dehydroxygenation with silane/BF₃, producing a novel series of core-modified ginkgolide analogs.

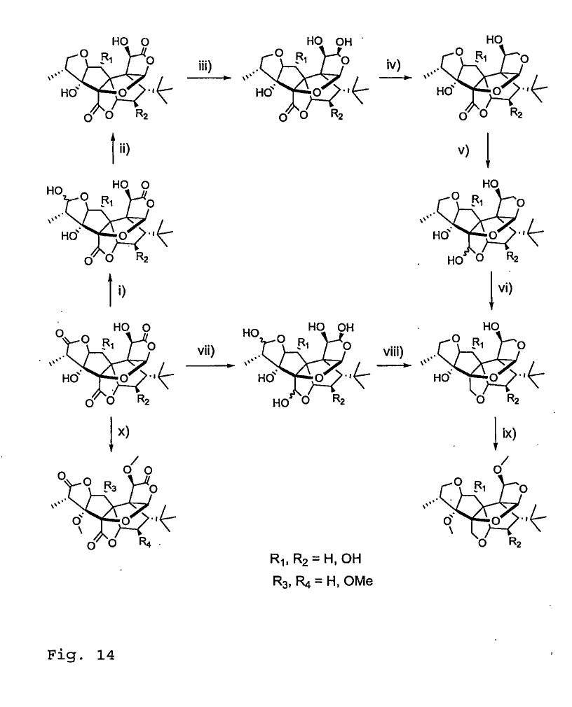

Further experiments confirmed the validity of the technique. As shown in the FIG. 14 structures produced by steps i) to vii), sequential removal of lactones was achieved. In addition, methylation of the C10 and C3 moieties sites was possible, FIG. 14, structures produced by steps ix), x), xi) and xii).

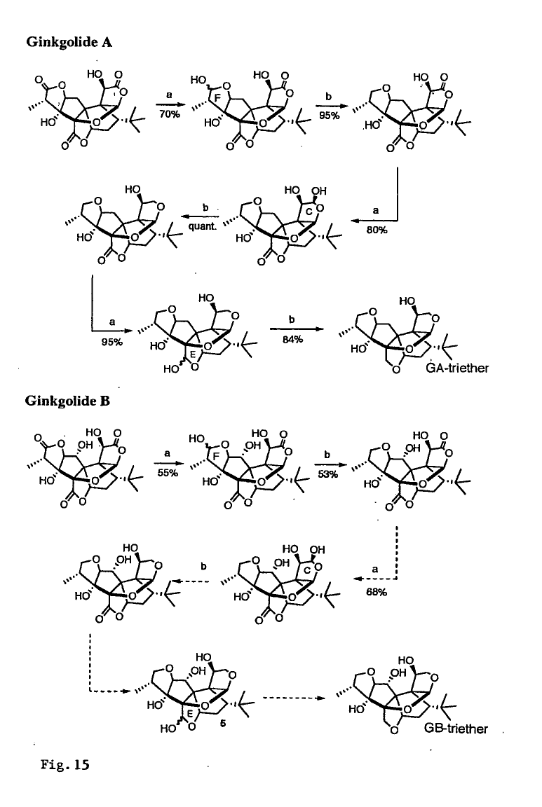

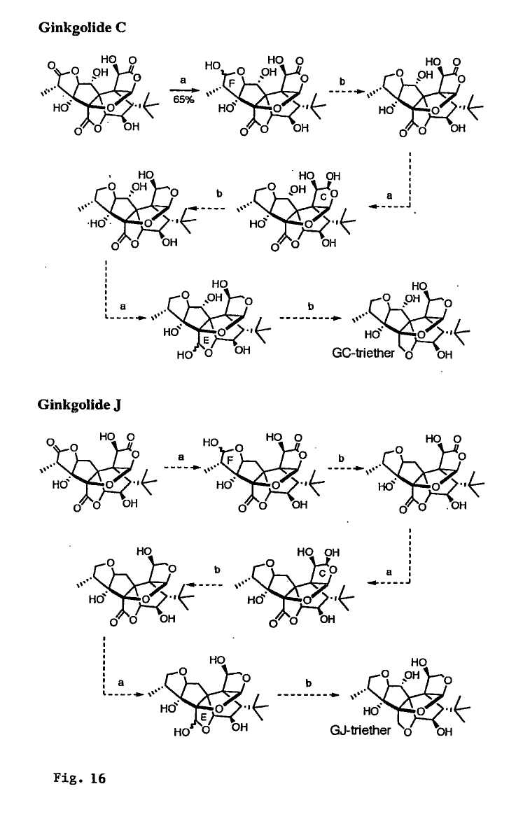

The step-wise transformation of ginkgolides A, B, C, and J to the corresponding “triethers” is shown in FIGS. 15 and 16.

### Methods and Materials

In a typical experimental procedure, GA (64.5 mg, 0.158 mmol) was dissolved in dry THF (4.0 ml), cooled to −78° C. under argon and 0.50 ml of DIBAL-H (1M solution in dichloromethane or hexanes) was added. The mixture was allowed to stir for two hours, warmed to room-temperature and EtOAc (11.0 ml) was added, followed by 3N HCl (0.3 ml) and water (5.0 ml). The mixture was extracted with EtOAc (3×20 ml). Organic phase was separated, washed with brine (3×20 ml), dried (Na₂SO₄), and solvent removed under vacuum. The lactol 1 (50.3 mg, 78% yield) was isolated as white solid by preparative TLC (hexane/acetone-1/1) as a ca. 3:2 mixture of diastereomeres: ¹H NMR (300 MHz, MeOH-d₄): major isomer, δ 5.69 (s, 1H), 5.35 (d, J=5.0 Hz, 1H), 4.96 (s, 1H), 4.78 (d, J=3.4 Hz, 1H), 4.63 (t, J=7.7 Hz, 1H), 2.56 (m, 2H), 2.17 (m, 2H), 1.89 (m, 2H), 1.09 (m, 12H); HRMS (FAB) m/z calcd for C₂₀H₂₉O₉Na 433.1475. found 433.1494. Lactol 1 (50.3 mg, 0.123 mmol) was dissolved in CH₂Cl₂ (6.0 ml), cooled to −78° C., and Et₃SiH (0.098 ml, 0.61 mmol) was added, followed by BF₃.Et₂O (0.039 ml, 0.304 mmol). The reaction mixture was warmed to room temperature over 12 h, quenched with saturated NaHCO₃ (1.0 ml) and water (5.0 ml) and subsequently extracted with EtOAc (3×20 ml). Organic layer was separated, washed with brine (3×20 ml), dried (Na₂SO₄) and solvent removed under vacuum. 2 (45.3 mg, 93% yield) was isolated by preparative TLC (hexane/acetone—1/1). ¹H NMR (300 MHZ, MeOH-d₄): δ 5.97 (s, 1H), 4.97 (s, 1H), 4.75 (d, J=3.4 Hz, 1H), 4.41 (t, J=7.8 Hz, 1H), 4.18 (t, J=7.9 Hz, 1H), 3.63 (dd, J=10.5, 8.0 Hz, 1H), 2.80 (m, 1H), 2.45 (dd, J=14.9, 7.0 Hz, 1H), 2.15 (m, 2H), 2.02 (dd, J=15.0, 8.0 Hz, 1H), 1.86 (dd, J=13.3, 5.6 Hz, 1H), 1.08 (s, 9H), 1.03 (d, J=6.8 Hz, 3H); ¹³C NMR (MeOH-d₄): δ 8.5, 28.5, 32.3, 36.3, 37.9, 38.8, 67.5, 69.5, 69.6, 76.2, 87.2, 89.4, 91.9, 110.7, 173.7, 175.2. HRMS (FAB) m/z calculated for C₂₀H₂₇O₈ 395.1706. found 395.1707.

Materials and Methods: All reagents were used as received. Ginkgolides were isolated from Ginkgo biloba extract (BioGinkgo 7/27, Pharmanex®. All reactions were conducted under argon in dry solvents and the yields refer to isolated products. Reactions were monitored by TLC (silica gel 60 F₂₅₄) and spots were visualized by heating and UV (or 12). Preparatory TLC was performed using silica gel MERCK5715 plates. Column chromatography was performed using silica gel (230-400 mesh). ¹H NMR and ¹³C NMR were recorded on Bruker (300 or 400 MHz) spectrometers. The chemical shifts are reported in ppm (δ) downfield from tetramethylsilane (in CDCl₃) or calibrated to solvent residual peak as an internal standard (MeOH-d₄, δ 3.31). High-resolution mass spectra (HRMS) were measured on JEOL JMS-HX110/100A HF mass spectrometer under FAB conditions with NBA as a matrix.

General procedure A—reduction with DIBAL-H: GA (64.5 mg, 0.158 mmol) was dissolved in dry THF (4.0 ml), cooled to −78° C. under argon and 0.5 ml of DIBAL-H (1M solution in dichloromethane or hexanes) was added. The mixture was allowed to stir for two hours, warmed to room-temperature and EtOAc (11.0 ml) was added, followed by 3N HCl (0.3 ml) and water (5.0 ml). The mixture was extracted with EtOAc (3×20 ml). Organic phase was separated, washed with brine (3×20 ml), dried (Na₂SO₄), and solvent removed under vacuum. GA-diether-F-lactol (3:2 mixture of diastereomeres) was isolated as a white solid by preparative TLC (hexane/acetone-1/1).

General procedure B—deoxygenation with Et₃SiH/BF₃.Et₂O: GA-diether-F-lactol (50.3 mg, 0.123 mmol) was dissolved in CH₂Cl₂ (6.0 ml), cooled to −78° C., and Et₃SiH (0.098 ml, 0.61 mmol) was added, followed by BF₃.Et₂O (0.039 ml, 0.304 mmol). The reaction mixture was warmed to room temperature over 12 h, quenched with saturated NaHCO₃ (11.0 ml) and water (5.0 ml) and subsequently extracted with EtOAc (3×20 ml). Organic layer was separated, washed with brine (3×20 ml), dried (Na₂SO₄) and solvent removed under vacuum. GA-diether was isolated by preparative TLC (hexane/acetone-1/1).

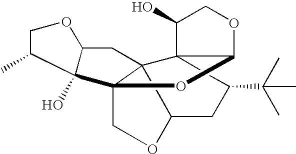

GA-triether: Starting from GA-trilactol: GA-trilactol (3.1 mg, 7.5 μmol) was suspended in CH₂Cl₂ (2 ml) followed by the addition of Et₃SiH (15.3 mg,). The mixture cooled to −78° C. and BF₃.Et₂O (14.2 mg) was added, and stirring at room temperature continued for 11 h. NaHCO₃ sat. (0.1 ml) was added followed by H₂O (5.0 ml) and EtOAc (10 ml). The layers were separated and aqueous phase was washed with EtOAc (2×10 ml). Organic fractions were combined and washed with brined (3×10 ml), dried (Na₂SO₄), and volatiles removed in vacuum. GA-triether (2.3 mg, 84% yield) was isolated by prep-TLC (hexane/acetone—1/1) as a white solid. Starting from Gatriether-E-lactol: GA-triether was obtained according to general procedure B in 84% yield. ¹H NMR (CDCl₃): 5.58 (s, 1H), 5.02 (t, J=8.9 Hz, 1H), 4.37 (t, J=7.8 Hz, 1H), 4.31 (d, J=10.5 Hz, 1H), 4.18 (t, J=8.0 Hz, 1H), 4.00 (m, 2H, 3.85 (d, J=10.5 Hz, 1H), 3.68 (dd, J=11.2, 7.8 Hz, 1H), 3.04 (m, 1H), 2.55 (dd, J=14.9, 9.1 Hz, 1H), 2.30 (dd, J=13.4, 5.4 Hz, 1H), 2.10 (dd, J=14.9, 7.5 Hz, 1H), 1.91 (m, 2H), 1.09 (s, 9H), 1.01 (d, J=6.5 Hz, 3H). ¹³C NMR (CDCl₃) 119.84, 108.92, 93.48, 93.11, 89.36, 77.85, 76.81, 74.74, 74.08, 72.08, 69.08, 53.13, 39.89, 38.07, 36.96, 33.39, 29.72, 9.71. HRMS (FAB) m/z: calcl for C₂₀H₃₁O₆: 367.2121. found 367.2110 [M+H].

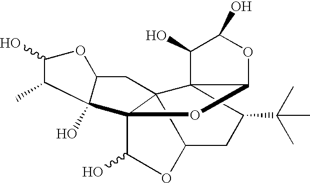

GA-trilactol: GA (20.1 mg, 49.6 μmol) was dissolved in THF (5 ml) and cooled to −78° C. 0.80 ml of DIBAL-H (1.0M in hexane) was added via syringe and the stirring continued for 3 h, followed by another 0.40 ml of DIBAL-H and stirring for another 3 h. The reaction mixture was brought to room temperature and 0.5 ml of 3N HCL was added, followed by H₂O (10 ml) and EtOAc (20 ml). Layers were separated, and the aqueous layer was washed with EtOAc (2×20 ml). Organic fractions were combined, washed with brine (3×20 ml) and dried over Na₂SO₄, and volatiles removed in vacuum. The residue was subjected to prep-TLC (hexane/acetone—1/2) to afford 2 (15.3 mg, 70% yield) as a colorless oil. HRMS (FAB) m/z: calcl for C₂H₂₆O₁₁Na: 465.1373. found 465.1383 [M+Na].

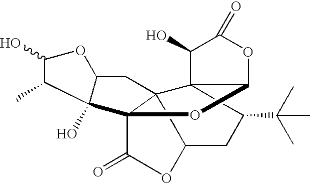

GA-F-lactol: Prepared from GA according to general procedure A in 70% yield as a white solid. ¹H NMR (300 MHz, MeOH-d₄): major isomer, 5.69 (s, 1H), 5.35 (d, J=5.0 Hz, 1H), 4.96 (s, 1H), 4.78 (d, J=3.4 Hz, 1H), 4.63 (t, J=7.7 Hz, 1H), 2.56 (m, 2H), 2.17 (m, 2H), 1.89 (m, 2H), 1.09 (m, 12H); minor isomer, 5.98 (s, 1H), 5.13 (d, J=7.7 Hz, 1H), 4.98 (s, 1H), 4.75 (d, J=3.5 Hz, 1H), 4.35 (dd, J=7.8, 7.1 Hz, 1H), 2.56 (m, 2H), 2.17 (m, 2H), 1.89 (m, 2H), 1.09 (m, 12H). HRMS (FAB) m/z: calcl for C₂₀H₂₆O₉Na: 433.1475. found 433.1494 [M+Na].

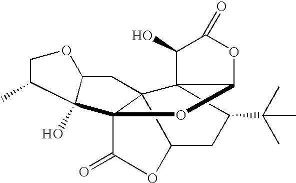

GA-diether: Prepared from GA-F-lactol GA-F-lactol according to general procedure B in 95% yield as a white solid. ¹H NMR (MeOH-d₄): 5.97 (s, 1H), 4.97 (s, 1H), 4.75 (d, J=3.4 Hz, 1H), 4.41 (t, J=7.8 Hz, 1H), 4.18 (t, J=7.9 Hz, 1H), 3.63 (dd, J=10.5, 8.0 Hz, 1H), 2.80 (m, 1H), 2.45 (dd, J=14.9, 7.0 Hz, 1H), 2.15 (m, 2H), 2.02 (dd, J=15.0, 8.0 Hz, 1H), 1.86 (dd, J=13.3, 5.6 Hz, 1H), 1.08 (s, 9H), 1.03 (d, J=6.8 Hz, 3H); ¹³C NMR (MeOH-d₄): 175.18, 173.66, 110.69, 91.89, 89.38, 87.15, 76.24, 69.60, 69.46, 67.54, 38.80, 37.93, 36.30, 32.27, 28.55, 8.51. HRMS (FAB) m/z: calcl for C₂₀H₂₇O₈: 395.1706. found 395.1707 [M+H]

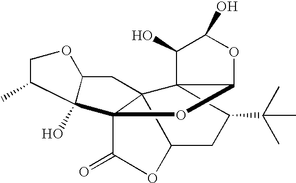

GA-diether-C-lactol: Prepared for GA-diether according to general procedure A in 80% yield as white solid. ¹H NMR (MeOH-d₄): 5.66 (s, 1H), 5.50 (d, J=5.2 Hz, 1H), 4.66 (d, J=3.2 Hz, 1H), 4.50 (d, J=5.2 Hz, 1H), 4.39 (t, J=6.8 Hz, 1H), 4.24 (t, J=7.9 Hz, 1H), 3.62 (dd, J=9.6, 8.0 Hz, 1H), 2.96 (m, 1H), 2.48 (dd, J=6.8, 3.3 Hz, 2H), 2.10 (m, 2H), 1.80 (dd, JI=13.3, 5.4 Hz, 1H), 1.10 (s, 9H), 1.01 (d, J=6.8 Hz, 3H). HRMS (FAB) m/z: calcl for C₂₀H₂₈O₈Na: 419.1682. found 419.1682 [M+Na]

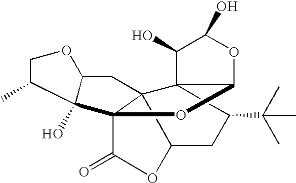

GA-triether: Prepared from GA-diether-C-lactol according to general procedure B, as a colorless oil in 100% yield. ¹H NMR (MeOH-d₄): 5.52 (s, 1H), 4.94 (t, J=8.9 Hz, 1H), 4.67 (m, 1H), 4.40 (t, J=6.6 Hz, 1H), 4.25 (t, J=7.9 Hz, 1H), 4.11 (t, J=8.7 Hz, 1H), 3.94 (t, J=8.2 Hz, 1H), 3.62 (dd, J=9.5, 8.1 Hz, 1H), 2.91 (m, 1H), 2.82 (dd, J=15.2, 6.7 Hz, 1H), 2.41 (dd, J=15.2, 6.6 Hz, 1H), 2.10 (m, 1H), 1.89 (m, 1H), 1.09 (s, 9H), 1.01 (d, J=6.8 Hz, 3H). ¹³C NMR (MeOH-d₄) 177.03, 118.94, 101.81, 93.28, 90.63, 89.30, 77.85, 73.79, 72.04, 71.44, 70.03, 39.22, 39.04, 37.11, 33.32, 29.56, 10.79. HRMS (FAB) m/z: calcl for C₂₀H₂₉O₇: 381.1913. found 381.1912 [M+H].

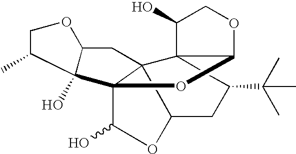

GA-triether-E-lactol: Prepared from GA-triether according to general procedure A in 95% yield as an oily solid. HRMS (FAB) m/z: calcl for C₂₀H₃₁O₇: 383.2070. found 383.2077 [M+H].

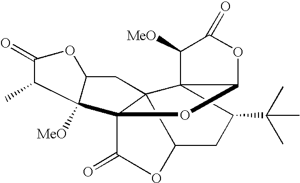

Dimethyl-GA: 0.15 g of KH in mineral oil was washed with hexane to give 50 mg of white solid, which was placed on dry ice under argon and GA (30.0 mg, 0.074 mmol) in 4.0 ml of THF was added dropwise and the reaction mixture was allowed to stir for 12 g at room temperature, before being quenched with H₂O (20 ml). The resulting solution was extracted with CH₂Cl₂ (3×20 ml), dried (MgSO₄) and volatiles removed in vacuum. The residue was washed with hexanes and dried in vacuum to afford 8 (14.7 mg, 45% yield) as a white solid. ¹H NMR (CDCl₃): 5.93 (s, 1H), 4.94 (dd, J=9.4, 7.6 Hz, 1H), 4.61 (d, J=3.7 Hz, 1H), 4.50 (s, 1H), 3.70 (s, 3H), 3.27 (s, 3H), 3.13 (q, J=7.1 Hz, 1H), 2.82 (dd, J=15.1, 7.4 Hz, 1H), 1.95 (m, 4H), 1.34 (d, J=7.1 Hz, 3H), 1.07 (s, 9H). HRMS (FAB) m/z: calcl for C₂₂H₂₉O₉: 437.1812. found 437.1819 [M+H].

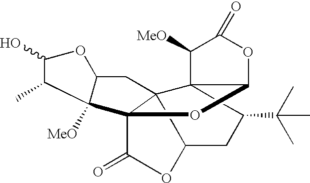

Dimethyl-GA-F-lactol: Prepared from dimethyl-GA according to general procedure A in 41% yield as an oily solid. ¹H NMR (CDCl₃): major 5.95 (s, 1H0, 5.37 (t, J=6.5 Hz, 1H), 4.75 (dd, J=9.3, 7.3 Hz, 1H), 4.57 (d, J=3.9 Hz, 1H), 4.50 (s, 1H), 3.71 (s, 3H), 3.38 (s, 3H), 3.03 (d, J=6.3 Hz, 1H), 2.80 (t, J=6.9 Hz, 1H), 2.58 (dd, J=14.8, 7.3 Hz, 1H), 2.20 (m, 4H), 1.21 (d, J=7.1 Hz, 3H), 1.06 (s, 9H); minor 5.92 (s, 1H), 5.39 (t, J=5.5 Hz, 1H), 5.04 (t, J=7.9 Hz, 1H), 4.63 (d, J=3.9 Hz, 1H), 4.48 (s, 1H), 3.70 (s, 3H), (s, 3H), 3.15 (d, J=9.3 Hz, 1H), 2.78 (m, 1H), 2.53 (dd, J=15.2, 7.4 Hz, 1H), 2.20 (m, 4H), 1.21 (d, J=7.1 Hz, 3H), 1.06 (s, 9H). HRMS (FAB) m/z: calculated for C₂₂H₃₁O₉: 439.1968. found 439.1953 [M+H]

### REFERENCES

(1) (a) Stromgaardt, K., Nakanishi, K. Angew. Chem. Int. Ed. 2004, 43, 1640-1658; (b) Stromgaard, K., Vogensen, S. B., Nakanishi, K. In Encyclopedia of Dietary Supplements, Marcel Dekker, 2005, pp 249-257.
(2) (a) Luo, Y., Smith, J. V., Paramasivam, V., Burdick, A., Curry, K. J., Buford, J. P., Khan, I., Netzer, W. J., Xu, H., Butko P. Proc. Nat. Acd. Sci., USA 2002, 99, 12197-12202; (b) Chromy, B. A., Nowak, R. J., Lambert, M. P., Viola, K. L., Chang, L., Velasco, P. T., Jones, B. W., Fernandez, S. J., Lacor, P. N., Horowitz, P., Finch, C. E., Krafft, G. A., Klein, W. L. Biochemistry 2003, 42, 12749-12760; (c) Yao, Z-X., Han, Z., Drieu, K., Papadoppulos V. J. Nutritional Biochem. 2004, 15, 749-756; (d) Lee, T-f., Chen, C-f., Wang, L. C. H. Phytotherapy Res. 2004, 18, 556-560.
(3) (a) Maruyama, M, A. Terahara, Y. Itagaki, K. Nakanishi, Tet. Lett. 1967, 4, 303-308; (b) Woods, M. C., Miura, I., Nakadaira, Y., Terahara, A., Maruyama, M., Nakanishi, K., ibid, 1967, 4, 321-326.
(4) Maione, A. M.; Quaglia, M. G. Chem. Ind., 1977, 6, 230.
(5) Marchand, A. P., Kumar, V. S., Hariprakasha, H. K., J. Org. Chem. 2001, 66, 2072-2077.
(6) (a) Kraus, G. A., Frazier, K. A., Roth, B. D., Taschner, M. J., Neuenschwander, K. J. Org. Chem. 1981, 46, 2417-2419; (b) Elbaum, D., Porco, J. A. Jr., Stout, T. J., Clardy, J., Schreiber, S. L. J. Am. Chem. Soc. 1995, 117, 211-225.

### Example 2

### GC to GJ Conversion

Ginkgolide J (GJ) has been shown to be very potent inhibitor of beta amyloid impairment of both long-term potentiation in electrophysiological studies, and of beta amyloid-induced cell death. Because of the scarcity of natural GJ in Ginkgo Biloba extract, a method was sought to produce GJ from the more abundant ginkgolide C (GC). Exposure of GC to an alkylating agent which can undergo a subsequent dexoygenation, as shown in FIG. 9A, in the presence of DMAP and a suitable solvent such as DMF resulted in the production of an alkylated intermediate. Subsequent deoxygenation by exposure of the intermediate to Et₃SiH, Bz₂O in toluene, resulted in the ultimate removal of 1-hydroxyl group from the starting ginkgolide. By this method, GJ was successfully made from GC (FIG. 9A).

Clearly this technique may be employed on all ginkgolides, and the resulting ginkgolides lacking hydroxyl groups are expected to be potent in terms of Alzheimer's therapy.

### Example 3

### Hydroxyl-Free Terpene Trilactones

The removal of the C10 hydroxyl from ginkgolide C described in Example 2 hereinabove prompted investigation of hydroxyl-free terpene trilactones. Using the same initial conditions, GA was converted to an alkylated intermediate, as shown in FIG. 9B. Subsequent deoxygenation by exposure of the intermediate to Et₃SiH, Bz₂O in toluene, and refluxing, resulted in the ultimate removal of the C10 and C3 hydroxyl groups. Conversion of the intermediate to the hydroxyl-free product could also by achieved by exposure to Bu₃SnH and AlBN (FIG. 9B).

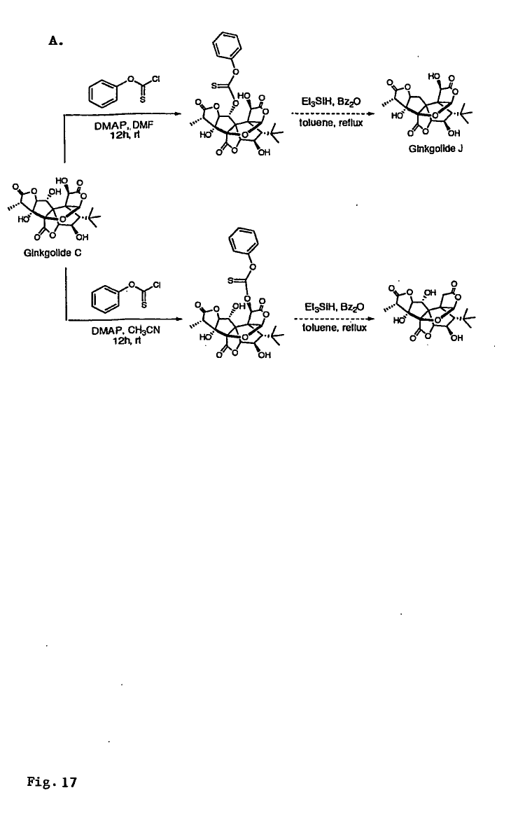

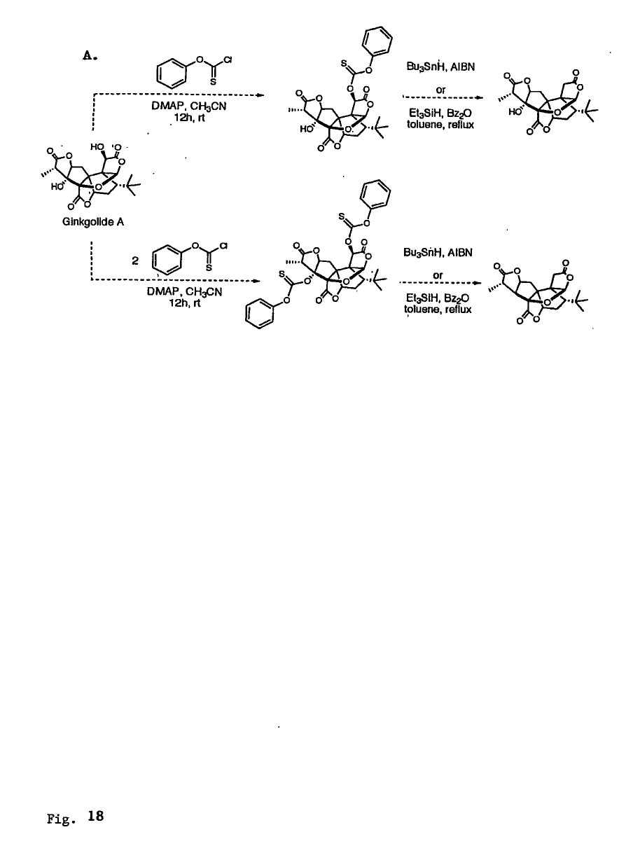

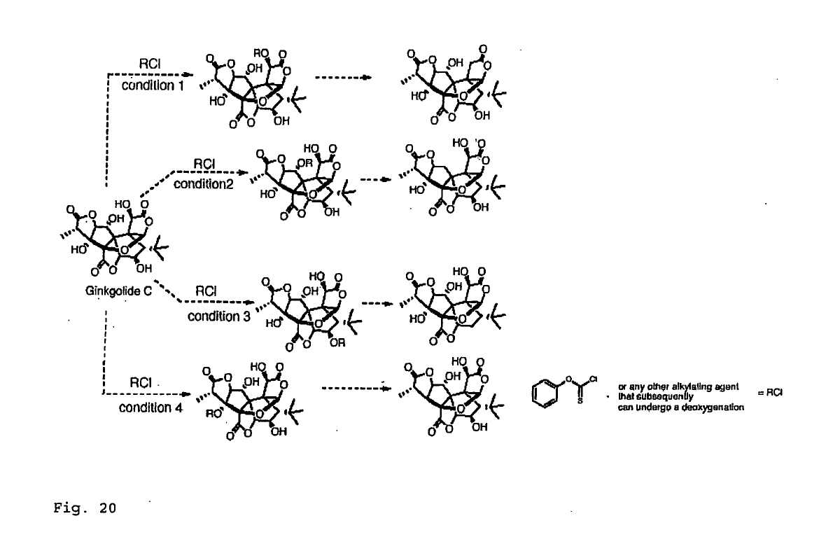

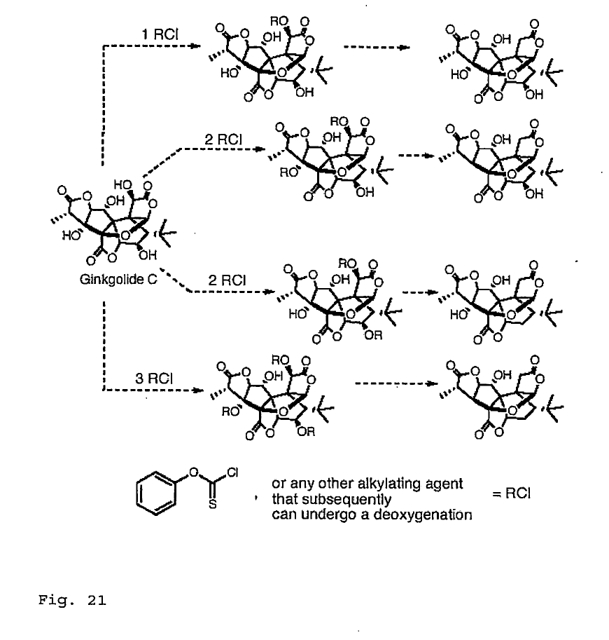

This technique (also shown for GC in FIG. 17A and GA in FIG. 18A) can be used to selectively functionalize ginkgolides at the hydroxyl positions (see FIGS. 20 and 21). Alkylating agents that can subsequently undergo a deoxygenation, such as thiochloromethoxy-benzene, may be used. Any RBr or RCl fitting this definition may be used.

The functionalization of C1 vs C10 with PhOC(S)Cl in the presence of the base (DMAP) is controlled by the solvent; DMF favors C1, whereas THF, CH₃CN favors C10. The derivatization of C7 takes place after either C1 or C10 functionalized and is not solvent dependent. To simultaneously place R groups at more than one of the C1, C7, C10, and C3 the amount of RCl or RBr is varied.

### Example 4

### Production of “Naked” Ginkgolides

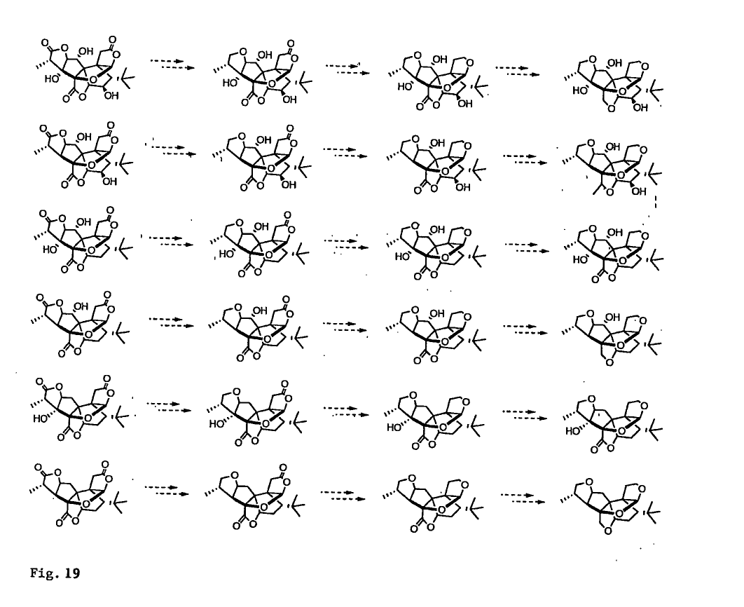

The production of hydroxyl free ginkgolides set a foundation for synthesizing “naked” ginkgolides, i.e. ginkgolides with one or more hydroxyls and lactones removed. FIG. 9C shows the removal of a lactone from the dehydroxylated GA product synthesized in Example 3 hereinabove. Exposure of the intermediate to DIBAL-H and then Et₃SiH and BF₃-ether as described hereinabove resulted in the production of a “naked” ginkgolide, stripped of both hydroxyls and lactones (FIG. 9C). FIG. 19 shows lactone-free ginkgolides based on GC, GB and GA starting ginkgolides.

### Example 5

### Effect of Ginkgolides on β-Amyloid Induced Impairment of Long-Term Potentiation and Cell Death

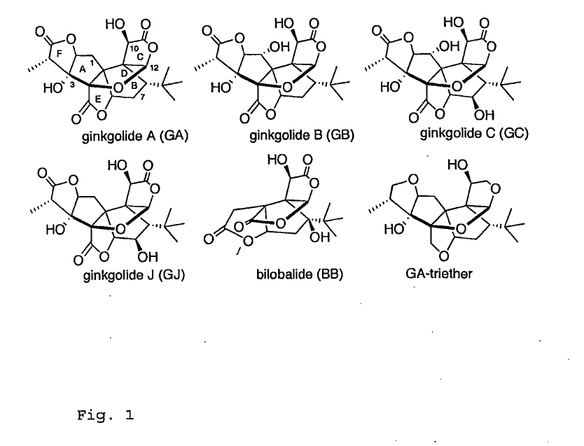

Extracts of the leaves and bark of the Ginkgo tree (Ginkgo biloba) have been promoted as memory enhancers in Asian traditional medicine for centuries. Globally, standardized extracts of Ginkgo leaves are among the largest selling herbal supplements accounting for sales of over 500 million dollars a year. In spite of this long experience, the number of well-controlled studies on the efficacy of these extracts is limited and its effect in the dementias appears to be modest in most cases (1-7). Extensive in vitro and in vivo studies appear to indicate that the extracts have potential efficacy in age-related cognitive decline or dementia. It is less likely that they affect memory in the non-demented (8). The results are interesting but have failed to lead to a consensus on the utilization of these extracts in treating dementia (9). A recent review of the literature finds that Ginkgo biloba extracts affect a variety of systems in the brain and that there is a need for further research on the effects Ginkgo biloba on learning and memory to encourage further research. However, it is questioned whether these results suggest a specific action of the extracts (10). The multiplicity of effects of Ginkgo biloba extracts on the brain could well be the result of the complex chemical composition of the extracts. The widely used and prescribed Ginkgo biloba extract, EGb-761, contains 6-7% terpene trilactones and 27% flavonoids, compounds with anti-oxidant activity, as well as a variety of other materials. While oxidative changes have been reported in Alzheimer's disease (AD), behavioral studies on APP transgenic mice show improvement in spatial memory in ginkgo treated mice in spite of unaltered amyloid levels and increasing levels of protein oxidation as compared to wild-type controls (11). These findings point to the terpene trilactones—the ginkgolides and bilobalide (FIG. 1) as the active compounds (12).

Here, the ability of Ginkgo biloba extract enriched 10 fold in terpene trilactones (P8A) as well as individual ginkgolides and bilobalide, and a ginkgolide derivative to reverse the amyloid beta (Aβ) induced inhibition of long-term potentiation (LTP) in the CA1 region of rat hippocampal slices and to block Aβ-induced cell death is investigated.

It was found that Ginkgo biloba extract, 70% enriched with terpene trilactones, prevents Aβ₁₋₄₂ induced inhibition of long-term potentiation in the CA1 region of rat hippocampal slices. This neuroprotective effect is attributed to ginkgolides A and J that completely replicate the effect of the extract. Ginkgolide J is also capable of inhibiting cell death of rodent hippocampal neurons caused by Aβ₁₋₄₂. This beneficial and multi-faceted mode of action of ginkgolide J makes it a new and promising lead in designing therapies against Alzheimer's disease.

LTP is an electrophysiological correlate of memory storage and is strongly inhibited by Aβ, the key neurotoxic agent in AD (13). The enriched TTL extract and two of the five individual ginkgolides tested (GA and GJ) as well as a derivative, GA-triether (The term GA-triether was originally used to indicate that three lactol-groups of GA are converted into the corresponding ether moieties, and does not intend to reflect the total number of ether rings in the molecule), blocked Aβ-induced depression of LTP. In addition, GJ blocked Aβ-induced cell death. These results point to a rational physiological basis for the use of these compounds in the treatment of dementia.

Earlier studies have shown that the inhibition of LTP by Aβ1-42 oligomers can be reversed by treatment with the phosphodiesterase 4 (PDE4) inhibitor rolipram (19). Recent studies have revealed that the Ginkgo biloba extract, EGb761, exerts PDE4 inhibitory activity with a pharmacological profile similar to that of rolipram (23,24). The possibility that Ginkgo biloba extracts might also block inhibition of LTP inhibition by Aβ1-42 oligomers was the premise for testing them on hippocampal slices. Based on a prior study suggesting that the effect of ginkgo extracts was likely to be mediated by the ginkgolides rather than by a decrease in oxidative damage mediated by the flavonoids (11), it was decided to test a new Ginkgo extract (P8A) that is 10-fold enriched in terpene trilactones and contains bilobalide and all four ginkgolides (GA, GB, GC, GJ) extracted from the leaves of the plant (14).

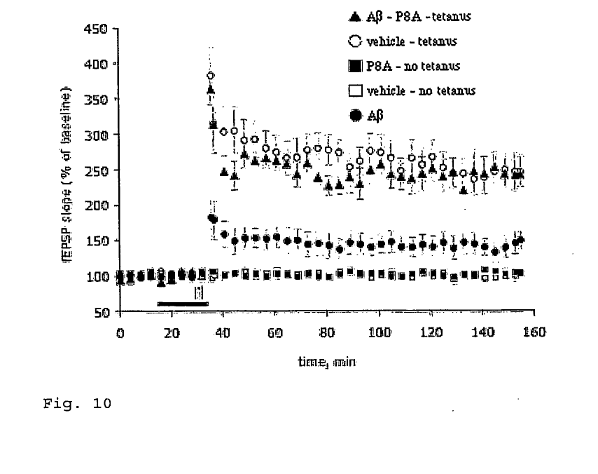

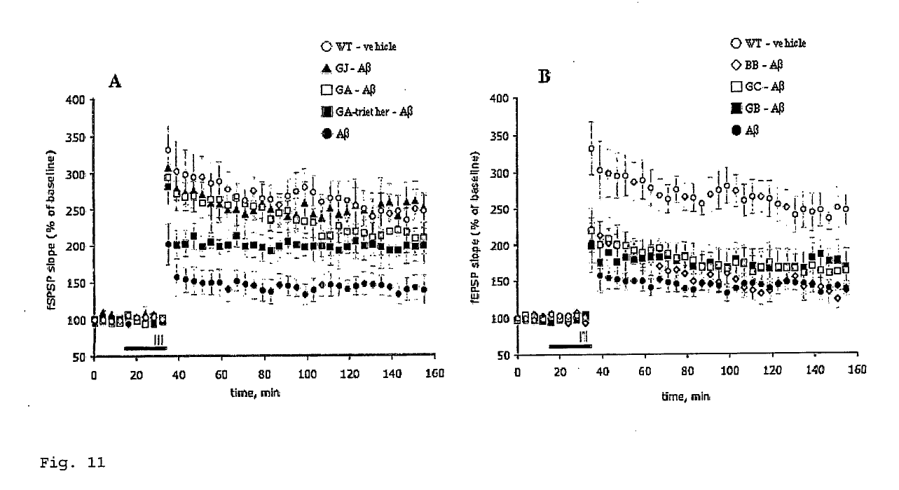

As shown in FIG. 10, treatment of hippocampal slices with 200 nM Aβ1-42 oligomers depressed LTP in the CA1 area to about a half of control values within 20 min of exposure. Treatment with P8A at 200 μg/ml was able to reverse the inhibition and restore LTP levels to control values. Neither Aβ nor P8A alone affected baseline transmission. Given the heterogeneity of molecules present in the extract, identification of the compounds that are responsible for the observed activity was attempted. In the same experimental setting, slices were co-treated with 200 nM Aβ1-42 oligomers and individual terpene trilactones (FIG. 3). GJ, GA, and GA-triether (at 1 μM each) were capable of reproducing the activity of the enriched extract and reversing Aβ-induced LTP impairment in CA1 region of hippocampal slices (FIG. 11A). A 20 min treatment with GJ, GA, or GA-triether rescues LTP impairment in slices treated with Aβ, although the efficiency of GA and GA-triether is slightly less than that of GJ, especially in case of GA-triether for the first 60 min after the tetanus. No effect was seen with GB, GC or BB (FIG. 11B).

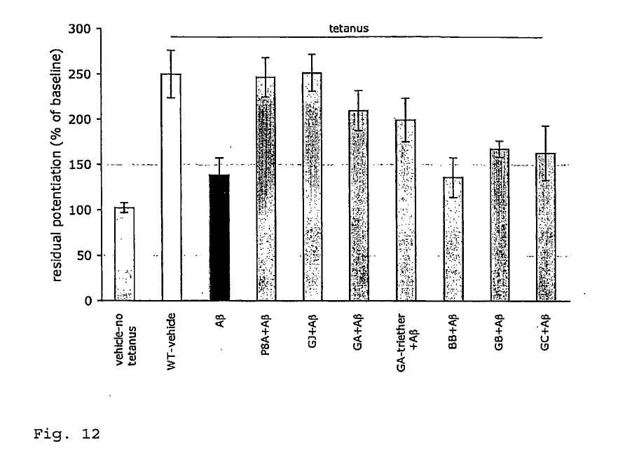

The results of electrophysiological experiments are summarized in FIG. 12, as amounts of potentiation at the end of the recording. It is evident that GJ is the most potent ginkgolide, completely mimicking the activity of the enriched extract.

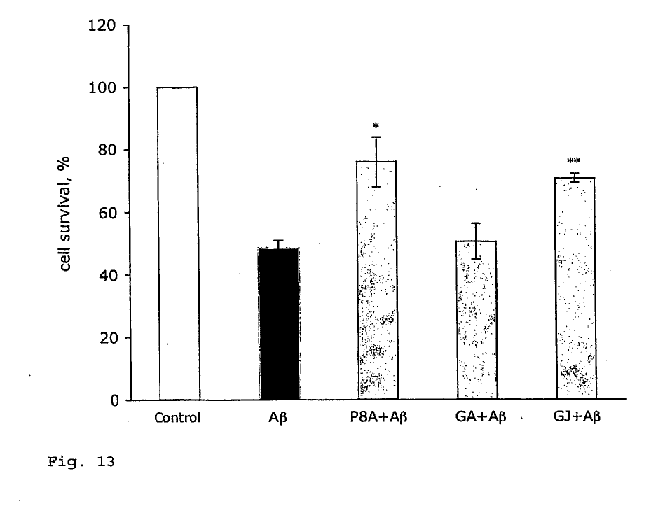

Neuronal cell death assay. In a parallel set of experiments we tested the ability of P8A and individual compounds (GA, GB, GC, GM, GJ, BB) to protect against cell death induced by a higher concentrations of oligomeric Aβ1-42 (FIG. 13). After a 24 hour exposure to 10 μM oligomeric Aβ1-42 only 48.5%±2.4 of cultured hippocampal cells survived. Addition of either 50 μg/ml of P8A or 1 μM GJ to the cultures at the same time greatly augmented survival with 76.0%±7.9 and 70.7%±1.4 of cells surviving respectively. A higher concentration of GJ (5 μM) completely prevented the Aβ-toxicity (data not shown). No improvement in the number of surviving cells was noted with GA (50.5%±+5.7) or the other ginkgolides and bilobalide even at high concentrations (data not shown). None of the substances affected neuronal viability when added alone (data not shown). These results are at variance with the studies of Bate et al. who demonstrated blockade of cell death with both GA and GB (25). The difference may be due to the fact that primary rodent hippocampal neurons were used in our studies while Bate et al. used the SY5Y human neuroblastoma cell line.

In the studies reported here, the effect of individual terpene trilactones (ginkgolides and bilobalide) on in two systems that are known to be affected by Aβ and thought to be related to AD—hippocampal long term potentiation and Aβ induced apoptosis was analyzed. The results appear to indicate that the extracts have potential efficacy in age-related cognitive decline or dementia. The present results demonstrate that both GJ and GA are capable of inhibiting the Aβ₁₋₄₂-induced damage to synaptic plasticity as reflected by LTP, but that only GJ can prevent cell death induced by higher concentrations of Aβ₁₋₄₂, suggesting that GJ may act also on an alternative pathway because of subtle structural differences from GA.

Collectively, these results demonstrate a very fine balance between the structure of the ginkgolide and its biological potential, and lead to preliminary structure-activity relationships: (a) the cage-like structure is required, as BB showed no activity; (b) the methylene group at the 1-position (FIG. 1) is essential for biological activity, i.e., GJ. GA, and GA-triether lack the 1-OH and exhibit activity, whereas GB and GC both with 1-OH-group are inactive (noteworthy, GB and GJ are regional isomers, yet the specific position of the hydroxyl-group determines the potency); (c) the presence of the hydroxyl-group at the 7-position does not seem to be crucial; (d) lactone-groups of native ginkgolides are important, but not essential, as GA-triether is still biologically active. Since only GJ shows neuroprotective properties in our studies, it is likely that the neuroprotective effects are mediated by a pathway different from that mediating synaptic effects. It also suggests that combinations of ginkgolides or their derivatives might be used in preventing memory loss and cognitive decline in Alzheimer's disease and related dementias by targeting different aspects of the disease process.

In summary, the enriched extract can completely prevent the detrimental effect of Aβ on LTP. Furthermore, the effects of the extract on LTP can be replicated by some of the individual ginkgolides, pointing for the first time to GJ as the most potent compound of the extract. The results clearly show that at least some of the biological effects of Ginkgo biloba extracts can be attributed to the individual terpene trilactones and that their use, as well as the use of their derivatives, might lead to more effective therapy.

### Materials and Methods

All chemicals were purchased from Sigma-Aldrich. Aβ₁₋₄₂ was purchased from American Peptide.

Ginkgo extracts and ginkgolides—Preparation of 70% enriched terpene trilactone fraction, P8A: In brief, a commercial extract of Ginkgo biloba leaves (Bioginkgo 7/27®) was boiled with 3% hydrogen peroxide to prevent the formation of emulsions that hindered efficiency of subsequent extractions. This was followed by extraction with ethyl acetate, washing with basic solutions, and charcoal filtration yielding an off-white powder with terpene trilactone content of 70% (14).

Isolation of Ginkgolides and Bilobalide—The individual compounds were isolated and characterized as previously described (12,15). GA-triether was prepared according to published procedure (16,17). The individual ginkgolides were dissolved in DMSO and added to the culture medium at a ratio of 1:1000 (v/v), yielding a 0.1% DMSO solution.

Production of β-amyloid oligomers—Oligomeric Aβ₁₋₄₂ was prepared according to the method of Stine et al. (18). Lyophilized Aβ₁₋₄₂ was allowed to equilibrate at room temperature for 30 min to avoid condensation upon opening the vial. The lyophilized peptide was resuspended in 1,1,1,3,3,3-Hexafluoro-2-propanol (HFIP) to a concentration of 1 mM using a glass gas-tight Hamilton syringe with a Teflon plunger. HFIP was evaporated in a fume hood and the resulting clear peptide film was dried under vacuum (6.7 mTorr) in a SpeedVac (Savant Instruments). The desiccated pellet was stored at −20° C. Immediately prior to use the aliquots were resuspended to a final concentration of 5 mM in anhydrous dimethylsulfoxide (DMSO) by trituration in a pipette followed by bath sonication for 10 minutes. Aβ₁₋₄₂ (5 mM) in DMSO was diluted to 100 μM in ice-cold cell culture media, immediately vortexed for 30 seconds and incubated at 4° C. for 24 hours.

Slice preparation—Mice were decapitated, and their hippocampi were removed. Transverse hippocampal slices with a thickness of 400 μm were maintained in an interface chamber at 29° C., as previously described (19,20). They were perfused with saline solution (124.0 mM NaCl, 4.4 mM KCl, 1.0 mM Na₂HPO₄, 25.0 mM NaHCO₃, 2.0 mM CaCl₂, 2.0 mM MgSO₄, 10.0 mM glucose) continuously bubbled with 95% O₂ and 5% CO₂. Slices were permitted to recover for at least 90 minutes before recording.

Electrophysiological Recordings—fEPSPs were recorded from the CA1 region of the hippocampus by placing both the stimulating and the recording electrodes in CA1 stratum radiatum. BST was assayed by plotting the stimulus voltage (V) against slopes of fEPSP to generate input-output relations. For LTP experiments, baseline stimulation was delivered every minute at an intensity that evoked a response approximately 35% of the maximum evoked response. Baseline response was recorded for 15 minutes prior to beginning the experiment to assure stability of the response. LTP was induced using theta-burst stimulation (4 pulses at 100 Hz, with the bursts repeated at 5 Hz and each tetanus including 3 ten-burst trains separated by 15 seconds). P8A, GA, GB, GC, GJ, BB, GA-triether and vehicle in 0.1% DMSO were individually added to the bath solution for 20 min prior the induction of LTP at the same time as Aβ₁₋₄₂.

Hippocampal neuronal culture—Hippocampal cell cultures were prepared according to the method previously described (21). Briefly, fetuses at embryonic day 18 (E18) from timed pregnant Sprague-Dawley rats (Taconic Farms) were sacrificed and the hippocampi removed. Neurons were then dissociated, plated at a density of 10⁶ cells/well on 6 well-plates coated with poly-L-lysine and maintained in a defined serum-free medium. The resultant cultures contained a population of cells enriched in the large pyramidal neurons that are a major target in AD. After 5-6 days in vitro (DIV) cells were used for the experiments.

Neuronal cell death assay—Hippocampal cultures were treated by adding 10 μM Aβ₁₋₄₂ in its oligomeric form with or without P8A at 50 μg/ml, or alternatively each of the individual ginkgolides and bilobalide (GA, GB, GC GJ, BB) at a concentration of 1 μM. After 24 h the number of viable cells was assessed by nuclear counting (22). Values represent mean±SEM of three consecutive experiments. Each experiment was performed in triplicate.

### Example 6

### Solvent Influence on Regioselectivity of Functionalization

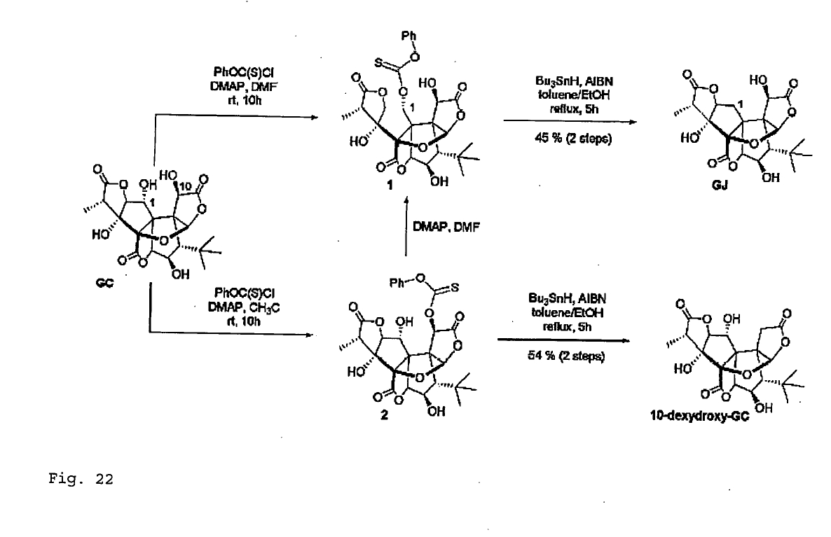

To develop the preparation of GJ, we envisioned a selective functionalization of 1-hydroxyl group of GC, which can be subsequently deoxygenated. The presence of three secondary hydroxyl groups in the GC structure might require the use of protecting groups to achieve the desired functionalization. In general, regioselectivity of GC functionalization depends on the nature of the electrophile, base and solvent. It was previously shown that GC can be selectively silylated into 1-position in DMF (26). The 10-OH is primarily functionalizied upon alkylation or acetylation of GC in different solvents and in the presence of different bases (27), whereas reaction with Tf₂O in pyridine yields 7-OTf GC (28). In our study, thionocarbonation of GC with O-phenylchlorothionoformate in the presence of 2.0 equivalents of DMAP in DMF led to the desired 1-thionocarbonylated GC 1 as the major product in good yield; whereas in acetonitrile the reaction yielded thionocarbonate 2 (FIG. 22). Lesser amounts of DMAP resulted in inferior yields of thionocarbonates. Deoxygenation reaction under standard Barton-McCombie conditions of these 1- and 10-thionocarbonates afforded GJ and the new deoxygenated ginkgolide 10-dehydroxy-GC, respectively.

Based on the results of GC thionocarbonation, we explored the effect of solvent on regioselectivity and on the conversion of GC into 1 in more detail (Table 1). Similar to acetonitrile (entry 1), dioxane and EtOAc also led to formation of 10-substituted product, thionocarbonate 2 as the main product. Selectivity was completely lost when reaction was performed in either THF or N,N dimethylacetamide (entries 4 and 5, respectively), whereas pyridine favored the formation of 1 (entry 6). DMF was the only solvent that selectively yielded thionocarbonate 1 (entry 7). Next, effect of bases on the regioselectivity of thionocarbonation in DMF was investigated using DMAP, pyridine, N-methylimidazole and Et₃N (entries 7-10, respectively). The effect of Hunig base, iPr₂EtN, was not studied here, since it was shown previously that in the presence of this base, GC rearranges into iso-GC. The selectivity was low with pyridine (entry 8). However, in the case with N-methylimidazole and Et₃N, the regioselectivity switched, leading to the formation of thionocarbonate 2 as the major product. Thus, as evident from Table 1, DMF/DMAP (entry 7) was the only combination that led to almost exclusive formation of 1, which is the desired intermediate in the preparation of GJ.

TABLE 1
Effect of base/solvent on the
regioselective thiocarbonylation of GC^{a}

| Entry | Solvent | Base | 1:2 (conversion, %)^{b} |
|---|---|---|---|
| 1 | CH3CN | DMAP | 1:15 (85) |
| 2 | Dioxane | DMAP | 1:12 (66) |
| 3 | EtOAc | DMAP | 1:2 (50) |
| 4 | THF | DMAP | 1:1 (61) |
| 5 | NNDA^{c} | DMAP | 1:1 (60) |
| 6 | Pyridine | DMAP | 4:1 (35) |
| 7 | DMF | DMAP | 10:1 (90) |
| 8 | DMF | Pyridine | 2:1 (74) |
| 9 | DMF | N-Methylimidazole | 1:6 (57) |
| 10 | DMF | Et3N | 1:4 (67) |

^{a}Reaction conditions: GC (20.0 mg, 0.045 mmol), PhOC(S)C1 (12.0 μL, 0.099 mmol), base (0.090 mmol), solvent (0.30 ml), 10 h, rt;
^{b}Estimated by ¹H NMR of the crude mixture.
^{c}N,N dimethilacetamide.

In the course of this study, we also found that the regioselectivity of thionocarbonation reaction is extremely sensitive to the amount of DMAP (Table 2).

As shown in Table 2, increasing the number of equivalents of DMAP favors the formation of thionocarbonate 1 regardless of the solvent used. These results prompted us to propose that thionocarbonate 2 can be transformed into 1 upon treatment with DMAP. Indeed, the formation of 1 was observed when 2 was treated with DMAP (2.2 eq.) in DMF for 3 h at room temperature (FIG. 22), which is in agreement with the data presented in Table 2. Likewise, 1 was treated with DMAP (2.2 eq.) in CH₃CN—the formation of 2 was not detected after 3 h. The thionocarbonylation protocols were also applied to other ginkgolides. Thionocarbonation of GA in the presence of DMAP in both DMF and CH₃CN yielded no products, and unreacted GA was recovered in both cases, thus supporting the proposed interplay between 1- and 10-hydroxy groups (27). Functionalization of GB happened to be less efficient with respect to regioselectivity and conversion, as compared to GC and, therefore, was not pursued further.

TABLE 2
Effect of DMAP on the regioselective thiocarbonylation of
GC^{a}

| Entry | Solvent | Equivalents of DMAP | 1:2^{b} |
|---|---|---|---|
| 1 | DMF | 1.5 | 2:1 |
| 2 | DMF | 2.0 | 10:1 |
| 3 | DMF | 5.0 | 10:1 |
| 4 | CH₃CN | 2.0 | 1:15 |
| 5 | CH₃CN | 5.0 | 1:3 |

^{a}Reaction conditions: GC (20.0 mg, 0.045 mmol), PhOC(S)C1(12.0uL, 0.099 mmol), solvent (0.30 ml), 10 h, rt;
^{b}Estimated by ¹H NMR of the crude mixture.

In conclusion, we have demonstrated that regioselectivity of GC thionocarbonation could be controlled by solvent as well as the amount and identity of the base. DMF and DMAP were efficient in promoting the formation of 1-substituted product 1, whereas the use of CH₃CN in combination with DMAP exclusively afforded 10-substitued product 2. Deoxygenation of thionocabonates 1 and 2 afforded GJ and the novel analog 10-dehydroxy-GC, respectively. The latter compound should be a useful model to probe the contribution of this hydroxyl group in ginkgolide-receptor interactions, since all native ginkgolides carry a hydroxyl group at C-10.

### Materials and Methods

GC to GJ conversion (typical experimental procedure): A mixture of GC (100 mg, 0.23 mmol) and DMAP (56 mg, 0.46 mmol) was dissolved in DMF (1.5 ml) under argon, and PhOC(S)Cl (60 μL, 0.51 mmol) was added via syringe under vigorous stirring. The reaction mixture was allowed to stir for 10 hours at room temperature, quenched with water (50 ml), 1N HCl (3.0 ml), and washed with EtOAc (3×100 ml). The organic fractions were combined and washed with brine (3×20 ml), dried (MgSO₄) and volatiles removed in vacuum. The residue was subjected to column chromatography (1:1-hexane:EtOAc) to afford 1 (93 mg, 70% yield); ¹H NMR (400 MHz, CD₃OD) δ 7.49-7.41 (m, 2H), 7.35-7.28 (m, 1H), 7.18-7.11 (m, 2H), 6.07 (s, 1H), 6.00 (d, J=5.0 Hz, 1H), 5.15 (d, J=4.2 Hz, 1H), 5.12 (s, 1H), 4.92 (d, J=5.0 Hz), 4.32 (dd, J1=12.3 Hz, J2=4.2 Hz, 1H), 3.09 (quart, J=7.3, 1H), 1.79 (d, J=12.4 Hz, 1H), 1.26 (d, J=7.3 Hz, 3H), 1.21 (s, 9H); ¹³C NMR (75 MHz, CD₃OD) δ 194.0, 177.7, 174.3, 172.2, 155.0, 130.7, 127.8, 122.9, 110.8, 101.3, 93.2, 85.3, 84.5, 81.6, 75.8, 70.1, 67.5, 66.4, 60.0, 42.7, 33.4, 29.6, 9.2. 1 (23 mg, 0.04 mmol) was dissolved in 1.1 ml of toluene:EtOH (9:1) mixture under argon, followed by the addition of AlBN (ca. 1 mg) and Bu₃SnH (42 μL, 0.16 mmol). The mixture was refluxed for 5 h, and then passed through a short KF containing silica gel column, concentrated and subjected to flash chromatography (hexane:EtOAc—2:1) to give GJ (10 mg, 60% yield), whose spectral characteristics were identical to an authentic sample.

### Example 7

### Synthesis of Ginkgolide M

Ginkgolide M (GM), which is found only in the roots of the Ginkgo biloba tree and is an inhibitor of ligand-operated ion channels in the central nervous system, has been prepared in three steps from 10-benzylginkgolide C, an intermediate generated during the isolation and separation of ginkgolides from Ginkgo biloba leaf extract. The described synthetic sequence can be applied to access GM derivatives for biological studies.

Ginkgolides are believed to be responsible for a variety of neuromodulatory effects exhibited by G. biloba leaf extract, including learning and memory functions (29). Several studies addressed structure-activity relationships of ginkgolides toward platelet activating factor (30) and glycine (31) receptors and have indicated a very fine balance between the number and position of the hydroxy groups around the ginkgolide skeleton and biological activity.

It was recently demonstrated that ginkgolides could block and modulate the responses of several ion channel receptors (31). Noteworthy, GM, which is found only in the root of Ginkgo biloba L. (Ginkgoaceae), unlike other ginkgolides that are found in the leaves as well, was shown to be the most potent natural ginkgolide in blocking the responses of several receptor-gated channels, whereas other tested ginkgolides (GA, GB, GC, and GJ) showed antagonistic properties exclusively toward glycine receptor. In particular, of all the ginkgolides, GM exhibited the highest inhibition of the GABA_{A} receptor and efficiently displaced TBPS (35S-tert-butylbicyclophosphorothionate) from the convulsant binding site of GABA_{A}. Ion channel blocker properties make GM a lead for potential treatment of neurodegenerative disorders, such as Alzheimer's disease (32).

From a structural point of view, GM lacks the tertiary hydroxyl group at the C-3 position, which is present in other ginkgolides from G. biloba extract, and, therefore, represents a unique analogue to address the effect of subtle structural changes on ginkgolide receptor interactions. Yet the biological scope and potential of this ginkgolide is not broadly studied; apparently, the available quantities of GM are relatively small as compared to other ginkgolides, thus making structure-activity relationship studies quite challenging.

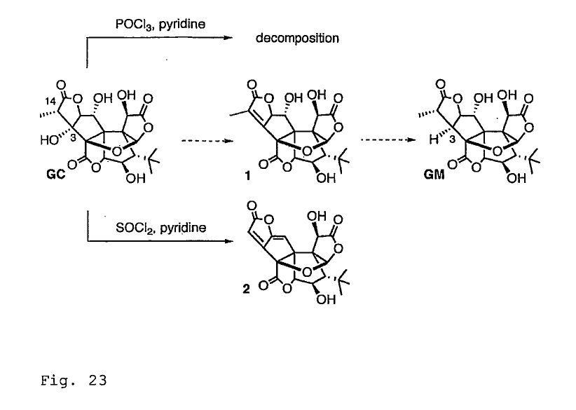

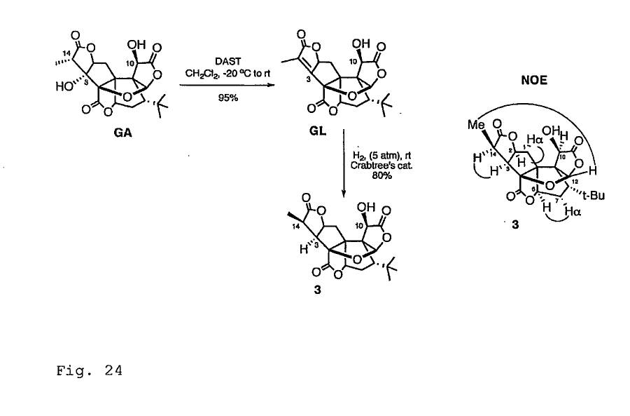

Therefore, a practical synthetic preparation of GM is desirable (see 32). Dehydration of OH-3 from unprotected GC would create a plausible intermediate, such as structure 1 in FIG. 23. en route to efficient synthesis of GM. However, subjection of GC to known dehydration procedures (32) led to either decomposition of the starting material or formation of the double-dehydrated product 2 of FIG. 23. see (33). During the course of studies directed toward regio-controlled synthesis of fluorinated ginkgolides, we found that treatment of GA with (diethylamino)sulfur trifluoride (DAST) (34) provided no fluorodehydroxylation at the C-10 position, but instead led to a high yield elective elimination of the tertiary hydroxy group, OH-3, affording GL (FIG. 24)(35). Upon hydrogenation of the unsaturated trilactone moiety of GL in the presence of Crabtree's catalyst (36), a clean formation of epi-derivative 3 took place (FIG. 24). The cis orientation of H-3 and H-14 in 3-dehydroxy-14-epi-GA, structure 3, was confirmed by NOE studies: an NOE was observed between the 14-Me and 12-H, which indicated that the 14-Me and 3-H are in a trans orientation; the 14-Me extends back toward the backbone, i.e., “\hat{α}-oriented” (FIG. 24). Further proof of stereochemistry is as follows: the 2D NOESY crosspeak volumes were analyzed using a NOE ratio method upon which the volume of the Me-14/H-12 cross-peak yielded a distance of 0.35 nm, consistent with the cis orientation of H-3 and H-14. The cis orientation of H-2 and H-3 was supported by NOESY volume analysis. The cross-peak volume between H-2 and H-3 was similar in magnitude to the peak volume for several other cis-oriented protons, such as between the H-6 and H-7R protons and between the H-2 and H-1R protons. For the NOE ratio method see 37.

Attempts to achieve dehydration of GC upon reaction with DAST under the conditions outlined in FIG. 24 led to decomposition of the starting material. (GB, however, was successfully converted into GK, using a DAST-mediated protocol). No desired elimination product was obtained when the reaction was conducted at different temperatures (−78 and 0° C.); instead, the starting material was recovered. Application of bases, i.e. pyridine and 4-(dimethylamino)pyridine (DMAP), to facilitate the dehydration process (38) was also unsuccessful.

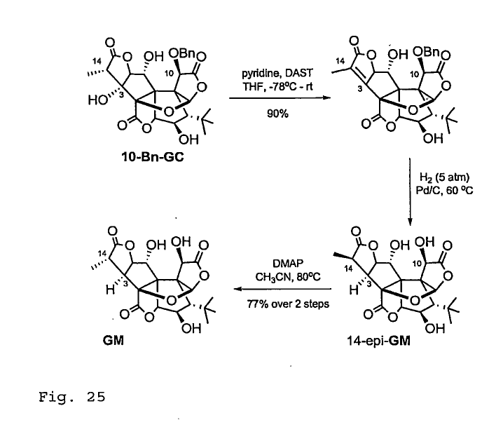

Since extra hydroxy groups of GC (as compared to GA and GB) are likely to contribute to the inefficiency of the dehydration, the monoprotected GC analogues were investigated. It is relevant to note that the 10-benzyl derivatives of GB and GC are intermediates prepared for the separation of individual ginkgolides from G. biloba leaf extract (39). Therefore, 10-benzyl-GC is an attractive starting point to explore the synthesis of ginkgolides and their derivatives. It was found that the reaction of 10-benzyl-GC 4 with DAST in the presence of pyridine in THF led to a clean elimination of the OH-3 group, giving unsaturated lactone 5 in good yield (FIG. 25). The DAST-mediated procedure appears to be quite general for the dehydration of ginkgolides and ginkgolide derivatives, as 10-benzyl-GB and 10-methyl-GC underwent a clean elimination of the OH-3 group, yielding the corresponding 3,14-unsaturated products in 90 and 85% yields, respectively.

Hydrogenation of the unsaturated lactone moiety yielded known 14-epi-GM, which in turn was converted into GM under previously reported conditions (32).

In conclusion, a short synthesis of GM has been achieved, which features selective removal of the tertiary hydroxy group in the presence of two unprotected secondary alcohol moieties and does not require the use of isolated GC. Thus the whole process can be achieved in a few steps starting from the commercial G. biloba leaf extract (see 40).

### Experimental Section

General Experimental Procedures. All reagents and solvents were purchased from Aldrich and used as received. Ginkgolides were available from earlier studies or isolated from BioGinkgo 27/7 extract according to a literature procedure (38 or 40). ¹H NMR spectra were recorded on a Bruker DPX-300 (300 MHz) spectrometer and are reported in ppm from CDCl₃ internal standard (7.26 ppm). 2D NOESY spectra were acquired on a Bruker DMX 500 spectrometer under the following conditions: TPPI mode; SW=3500 Hz; TD2=TD1*4=1200; D1=5 s; NS=40. For the NOESY volume ratio analysis, several different reference distances and NOEs were used, and the choice did not affect the calculated distances significantly.

Example of Dehydration Procedure. 10-Benzyl-GC 4 (15.0 mg, 0.029 mmol) was dissolved in 1 mL of THF and cooled to −78° C. Pyridine (100 μL, 1.23 mmol) and DAST (100 μL, 0.76 mmol) were added dropwise at this same temperature. The reaction mixture was stirred at −78° C. for 10 min, warmed to room temperature, and then kept for 40 min before quenching by addition of 2 mL of water. The aqueous layer was extracted with 3×2 mL of EtOAc. The combined organic layers were washed with brine, dried over sodium sulfate, and concentrated in vacuo. Purification by flash chromatography on silica gel (eluted with 1:1 EtOAc/hexane) afforded the desired 3,14-dehydro-10-benzyl-GC 5 (13.1 mg, 0.026 mmol, 90% yield) as a white solid: ¹H NMR (300 MHz, CDCl₃) δ 7.40 (5H, m), 6.00 (s, 1H), 5.85 (1H, s), 5.42 (1H, d, J) 10.2 Hz), 4.82 (1H, s), 4.63 (1H, d, J) 10.3 Hz), 4.60 (1H, d, J) 5.0 Hz), 4.39 (1H, m), 2.20 (3H, s), 2.03 (2H, m), 1.92 (1H, d, J) 12.4 Hz), 1.23 (9H, s).

Synthesis of GM. A glass liner for a stainless steel 45 mL Parr high-pressure reactor equipped with a stir bar was charged with a solution of 3,14-dehydro-10-benzyl-GC 5 (12.0 mg, 0.024 mmol) in a mixture of EtOAc (1 mL) and MeOH (3 mL), then Pd/C (10% w/w, 2 mg) was added. The liner was inserted into the Parr reactor, and the pressure gauge and gas assembly were attached. The reactor was sealed, charged and vented with 3×400 psi with H2, and recharged to 600 psi H₂. The reaction mixture was stirred at room temperature for 18 h, and then the reactor was vented. The solution was filtered through a pad of Celite and concentrated in vacuo. The residue was dissolved in 1 mL of MeCN, and DMAP (12.0 mg, 0.096 mmol) and 0.1 mL of water were added. The reaction mixture was stirred 80° C. in sealed tube for 4 days and then quenched by addition of 2 mL of 1 M HCl. The aqueous layer was extracted with 3×2 mL of EtOAc, and the combined organic layers were washed with brine, dried over sodium sulfate, and concentrated in vacuo. Purification by flash chromatography on silica gel (eluted with 2:1 EtOAc/hexane) afforded GM (7.9 mg, 0.019 mmol, 77% yield over two steps), whose spectral data matched those of the natural compound.

### Example 8

### Removal of Tertiary Hydroxyl

Removal of the tertiary OH group (i.e. the 3-OH) from ginkgolide and ginkgolide derivatives can be achieved by exposing the 3-OH-bearing ginkgolide or ginkgolide derivative to DAST and pyridine in a suitable solvent, as set forth in the following scheme:

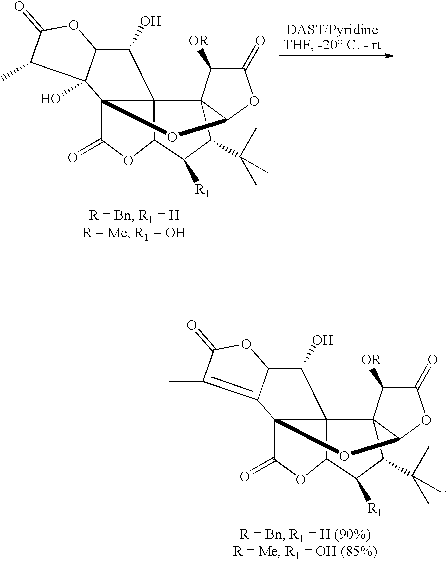

### Example 9

### Removal of Lactone Using Et₃Siallyl

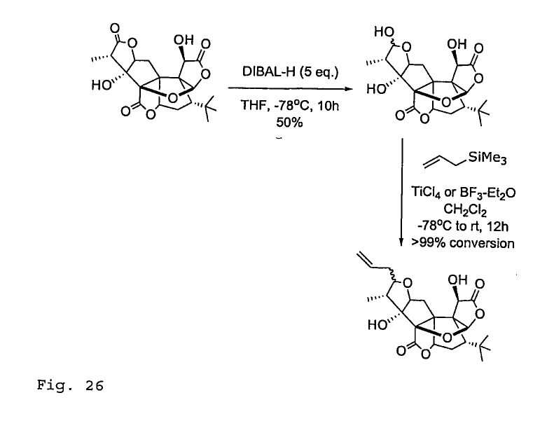

Lactones can be removed form the terpene trilactone cage skeleton or bilobalide using Et₃Siallyl (see FIG. 26). The allyl functionality replaces the lactone. This is clearly a very versatile way to functionalize ginkgolides since the allyl group can be easily derivatized into alcohol, acid, ester, etc. in addition to being a handle for numerous metathesis reactions. This introduction of an allyl group can be done on any of the free lactols of the ginkgolide skeleton, for example with GA, GB, GC, GM, GJ and bilobalide. An example of installing allyl-functionality on the F-ring is shown in FIG. 26.

### Example 10

### Functionalization of Ginkgolide B at the C10 Position

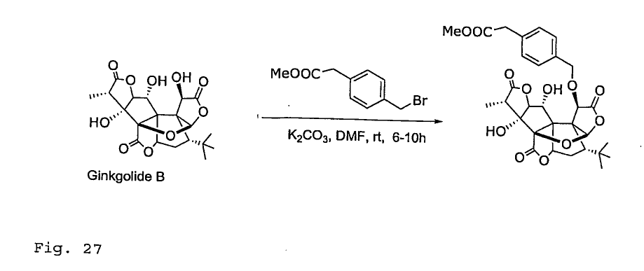

A scheme for functionalizing ginkgolide B at the C10 position is set forth in FIG. 27. K₂CO₃ can be substituted by other inorganic bases (e.g. NaH, KH, Na₂CO₃, etc) or organic bases (iPr2EtN, for example). DMF is usually the best solvent, but reaction proceeds in THF or CH₃CN, especially if NaH or KH are used a base. Reaction is not very efficient, the yield is about 10-15%, due to the cleavage of the ester group (MeOOC—) and the formation of the acid, which is converted into a salt, and leads to some precipitation of the reactant.

The reaction is expected to work with a similar efficiency with ginkgolide C, and less so with ginkgolides A and J in that it will require more stringent condition to obtain a product (heat, longer reaction times).

### Example 11

### Functionalization of Ginkgolide C at the C10 Position

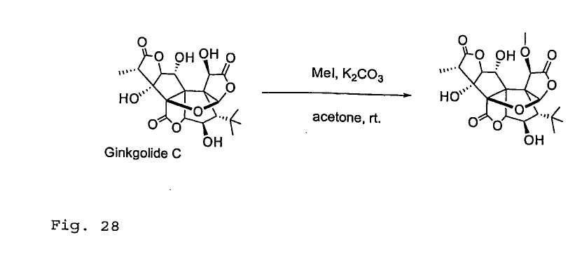

A scheme for functionalizing ginkgolide C at the C10 position is set forth in FIG. 28. The reaction proceeds under standard conditions; K₂CO₃ or NaH or KH as a base, and DMF or THF as a solvent may also be used. Due to high volatility of MeI, large amounts (10-20 eq.) of this reactant are employed. More ME groups may be introduced by large amounts of MeI.

The reaction also works for ginkgolide B quite efficiently. Less efficient synthesis was achieved for ginkgolide A using KH as a base. Ginkgolide J is expected to behave similar to ginkgolide A.

### Example 12

### Functionalization of Ginkgolide C at the C7 Position

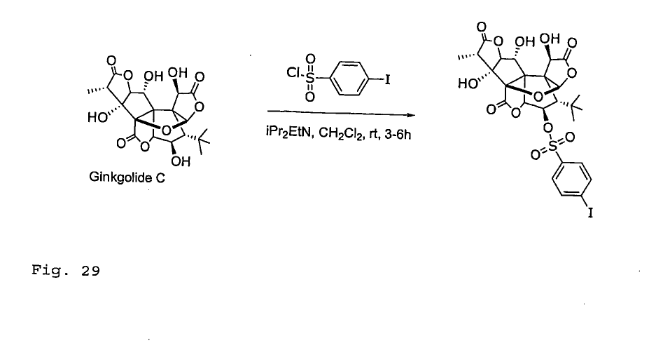

A scheme for functionalizing gingkolide C at the C7 position is set forth in FIG. 29. The solvent may be replaced with CHCl₃. Other organic bases (DMAP, pyridine, Et₃N, etc.) are expected to lead to functionalization at 7-position. Interestingly, in the presence of K₂CO₃ (inorganic base), the functionalization goes exclusively into 10-position. This procedure is expected to work for ginkgolide J also. Pyridine, CH₂Cl₂, −20 C for 2 h, rt for 1 h is also expected to achieve the same result.

### REFERENCES

1. Kanowski, S., Herrmann, W. M., Stephan, K., Wierich, W., and Horr, R. (1996) Pharmacopsychiatry. 29, 47-56
2. Le Bars, P. L., Kieser, M., and Itil, K. Z. (2000) Dement. Geriatr. Cogn. Disord. 11, 230-237
3. Maurer, K., Ihl, R., Dierks, T., and Frolich, L. (1997) J. Psychiatr. Res. 31, 645-655
4. Oken, B. S., Storzbach, D. M., and Kaye, J. A. (1998) Arch. Neurol. 55, 1409-1415
5. Rai, G. S., Shovlin, C., and Wesnes, K. A. (1991) Curr. Med. Res. Opin. 12, 350-355
6. Solomon, P. R., Adams, F., Silver, A., Zimmer, J., and DeVeaux, R. (2002) Jama. 288, 835-840
7. Wettstein, A. (2000) Phytomedicine 6, 393-401
8. Moulton, P. L., Boyko, L. N., Fitzpatrick, J. L., and Petros, T. V. (2001) Physiol. Behav. 73, 659-665
9. Doody, R. S., Stevens, J. C., Beck, C., Dubinsky, R. M., Kaye, J. A., Gwyther, L., Mohs, R. C., Thal, L. J., Whitehouse, P. J., DeKosky, S. T., and Cummings, J. L. (2001) Neurology 56, 1154-1166
10. Gold, P. E., Cahill, L., and Wenk, G. L. (2003) Sci. Am. 288, 86-91
11. Stackman, R. W., Eckenstein, F., Frei, B., Kulhanek, D., Nowlin, J., and Quinn, J. F. (2003) Exp. Neurol. 184, 510-520
12. Stromgaard, K., and Nakanishi, K. (2004) Angew. Chem. Int. Ed. Engl. 43, 1640-1658
13. Klein, W. L., Stine, W. B. J., and Teplow, D. B. (2004) Neurobiol. Aging. 25, 569-580
14. Lichtblau, D., Berger, J. M., and Nakanishi, K. (2002) J. Nat. Prod. 65, 1501-1504
15. Jaracz, S., Malik, S., and Nakanishi, K. (2004) Phytochemistry 65, 2897-2902
16. Maruyama, M., Terahara, A., Itagaki, Y., and Nakanishi, K. (1967) Tetrahedron Lett. 8, 303-308
17. Woods, M. C., Miura, I., Nakadaira, Y., Terahara, Y., Itagaki, Y., and Nakanishi, K. (1967) Tetrahedron Lett 8, 321-326
18. Stine, W. B., Jr., Dahlgren, K. N., Krafft, G. A., and LaDu, M. J. (2003) J. Biol. Chem. 278, 11612-11622
19. Gong, B., Vitolo, O. V., Trinchese, F., Liu, S., Shelanski, M., and Arancio, O. (2004) J. Clin. Invest. 114, 1624-1634
20. Vitolo, O. V., Sant'Angelo, A., Costanzo, V., Battaglia, F., Arancio, O., and Shelanski, M. (2002) Proc. Natl. Acad. Sci. USA 99, 13217-13221
21. Goslin, K., Asmussen, H., and Banker, G. (1998) Rat hippocampal neurons in low density culture, MIT Press, Cambridge
22. Troy, C. M., Rabacchi, S. A., Friedman, W. J., Frappier, T. F., Brown, K., and Shelanski, M. L. (2000) Neurosci. 20, 1386-1392
23. Campos-Toimil, M., Lugnier, C., Droy-Lefaix, M. T., and Takeda., K. (2000) Arterioscler. Thromb. Vasc. Biol. 20, E34-40
24. Saponara, R., and Bosisio, E. (1998) J. Nat. Prod. 61, 1386-1387
25. Bate, C., Salmona, M., and Williams, A. (2004) J Neuroinflammation. 1: http://www.jneuroinflammation.com/content/I/I/4.
26. Weinges, K., Schick, H., Liebigs Ann. Chem., 1991, 1, 81-83.
27. (a) Corey, E. J.; Rao, K. S.; Ghosh, A. K. Tetrahedron Lett., 1992, 33, 6955-6958; (b) Jaracz, S.; Stromgaard, K.; Nakanishi, K. J. Org. Chem. 2002, 67, 4623-4626.
28. Teng, B-p. Ger. Offen. 1992, DE 4212019, 6 pp.
29. (a) Nakanishi, K. Bioorg. Med. Chem. 2005, 13, 4983-5000 and (b)Stromgaard, K.; Nakanishi, K. Angew. Chem., Int. Ed. 2004, 43, 1640-1658.
30. (a) Hosford, D.; Mencia-Huerta, J. M.; Page, C.; Braquet, P. Phytotherapy Res. 1988, 2, 1-24. (b) Hu, L.; Chen, Z.; Xie, Y.; Jiang, Y.; Zhen, H. Bioorg. Med. Chem. 2000, 8, 1515-1521. (c) Vogensen, S. B.; Stromgaard, K.; Shindou, H.; Jaracz, S.; Suchiro, M.; Ishii, S.; Shimizu, T.; Nakanishi, K. J. Med. Chem. 2003, 46, 601-608. (d) Zhu, W.; Chen, G.; Hu, L.; Luo, X.; Gui, C.; Luo, C.; Puah, C. M.; Chen, K.; Jiang, H. Bioorg. Med. Chem. 2005, 13, 313-322.31. (a) Chatterjee, S. S.; Kondratskaya, E. L.; Krishtal, O. A. Pharmacopsychiatry 2003, 36 (Suppl. 1), S68-S77. (b) Ivic, L.; Sands, T. T. J.; Fishkin, N.; Nakanishi, K.; Kriegstein, A. R.; Stromgaard, K. J. Biol. Chem. 2003, 278, 49279-49285. (c) Jaracz, S.; Nakanishi, K.; Jensen, A. A.; Stromgaard, K. Chem. Eur. J. 2004, 10, 1507-1518. (d) Kondratskaya, E. L.; Betz, H.; Krishtal, O. A.; Laube, B. Neuropharmacology 2005, 49, 945-951.
31. Schmitt, H. P. Med. Hypothesis 2005, 65, 259-265.
32. (a) Weinges, K.; Rummler, M.; Schick, H. Liebigs Ann. Chem. 1993, 1023-1027. (b) Maruyama, M.; Terahara, A.; Nakadaira, Y.; Woods, M. C.; Takagi, Y.; Nakanishi, K. Tetrahedron Lett. 1967, 8, 315-319.
33. Maruyama, M.; Terahara, A.; Nakanishi, K. Sci. Rep. Tohoku Uni. 1967, 50, 92-99.
34. Middleton, W. J. J. Org. Chem. 1975, 40, 574-578.
35. Wang, Y.; Sheng, L. S.; Lou, F. C. Acta Pharmcol. Sin. 2001, 31, 606-608.
36. Crabtree, R. H. Acc. Chem. Res. 1990, 23, 95-101.
37. Newhaus, D.; Williamson, M. The Nuclear OVerhauser Effect in Structural and Conformational Analysis, 2nd ed.; Wiley-VCH: New York, 2000.
38. Somekh, L.; Shanzer, A. J. Org. Chem. 1982, 48, 907-908.
39. Jaracz, S.; Malik, S.; Nakanishi, K. Phytochemistry 2004, 65, 2897-2902.
40. Nakanishi, K.; Jaracz, S.; Malik, S.; Ishii, H.; Dzyuba, S. V. PCT Int. Appl. WO 2005046829, 2005, 61 pp.

1. A process of reducing a lactone or of replacing or removing a hydroxyl group on a terpene trilactone cage skeleton or a bilobalide comprising:
a) obtaining a lactone bearing terpene trilactone cage skeleton or bilobalide, or a hydroxyl bearing terpene trilactone cage skeleton or bilobalide, and
b) (i) exposing the lactone bearing terpene trilactone cage skeleton or bilobalide to DIBAL-H in a first suitable solvent to reduce the lactone and form a resulting compound having a hydroxyl group at the position of the lactone; or
(ii) exposing the hydroxyl bearing terpene trilactone cage skeleton or bilobalide to an alkylating agent capable of undergoing a subsequent deoxygenation, in the presence of DMAP and a second suitable solvent to form a first product and exposing the first product to Et₃SiH and Bz₂0 in the presence of a third suitable solvent or to Bu₃SnH and AlBN in the presence of a fourth suitable solvent, or exposing the hydroxyl bearing terpene trilactone cage skeleton or bilobalide to Et₃SiH and BF₃-Et₂0 in the presence of a fifth suitable solvent for a time sufficient to deoxygenate the hydroxyl group, or exposing the hydroxyl bearing terpene trilactone cage skeleton or bilobalide to an allylating agent and TiCl₄ or BF₃-Et₂0 in the presence of a seventh suitable solvent, so as to thereby replace the hydroxyl group on the terpene trilactone cage skeleton or bilobalide; or
(iii) exposing the hydroxyl bearing terpene trilactone cage skeleton or bilobalide to (diethylamino)sulfur trifluoride and pyridine in the presence of a sixth suitable solvent for a time sufficient to remove the hydroxyl group.

2. The process of claim 1, wherein the lactone bearing terpene trilactone cage skeleton is ginkgolide A, ginkgolide B, ginkgolide C, ginkgolide J, or ginkgolide M.

3. (canceled)

4. (canceled)

5. (canceled)

6. (canceled)

7. The process of claim 1, wherein the alkylating agent has the structure:

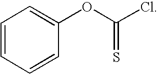

8. (canceled)

9. (canceled)

10. (canceled)

11. (canceled)

12. (canceled)

13. (canceled)

14. (canceled)

15. The process of claim 1, wherein in step b) (i) 4-5 equivalents of DIBAL-H are employed.

16. The process of claim 1, wherein in step b)(i) more than 20 equivalents of DIBAL-H are employed.

17. (canceled)

18. (canceled)

19. (canceled)

20. (canceled)

21. (canceled)

22. (canceled)

23. The process of claim 1, wherein the lactone bearing terpene trilactone cage skeleton is ginkgolide A which is reduced in step b(i) to:

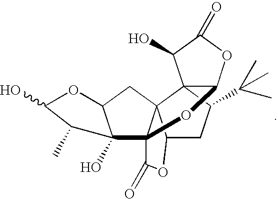

24. The process of claim 1, wherein the hydroxyl bearing terpene trilactone cage skeleton is reduced in step b)(ii) to:

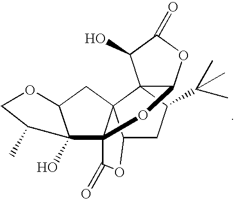

25. The process of claims 1, wherein the hydroxyl bearing terpene trilactone cage skeleton is reduced to form a first product having the structure:

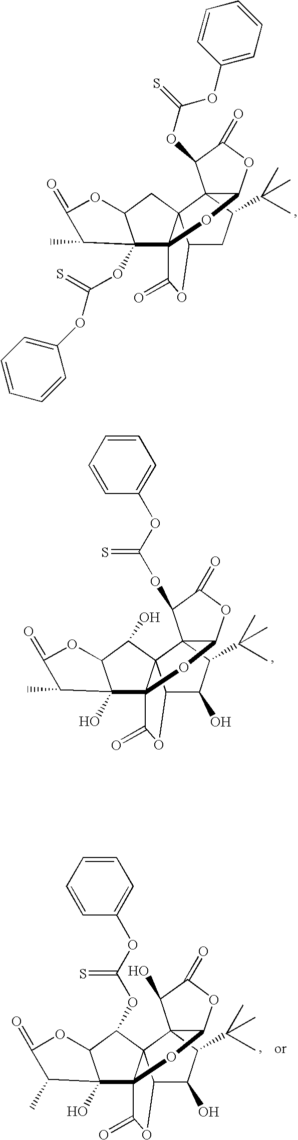

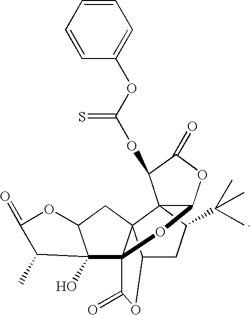

26. The process of claim 1, wherein the hydroxyl group of the hydroxyl bearing terpene trilactone cage skeleton is replaced to produce a compound having the following structure:

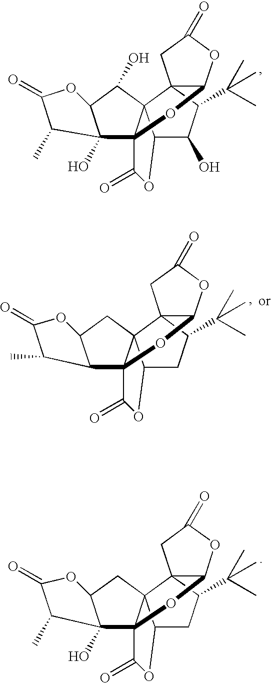

27. The process of claim 1, wherein step b) (i), step b)(ii), or step b)(i) and step b)(ii), are performed more than once on a single lactone bearing and/or hydroxyl bearing terpene trilactone cage skeleton.

28. (canceled)

29. (canceled)

30. (canceled)

31. (canceled)

32. The process of claim 1, wherein the terpene trilactone cage skeleton is reduced and/or has hydroxyl group(s) replaced to produce a compound having one of the following structures: 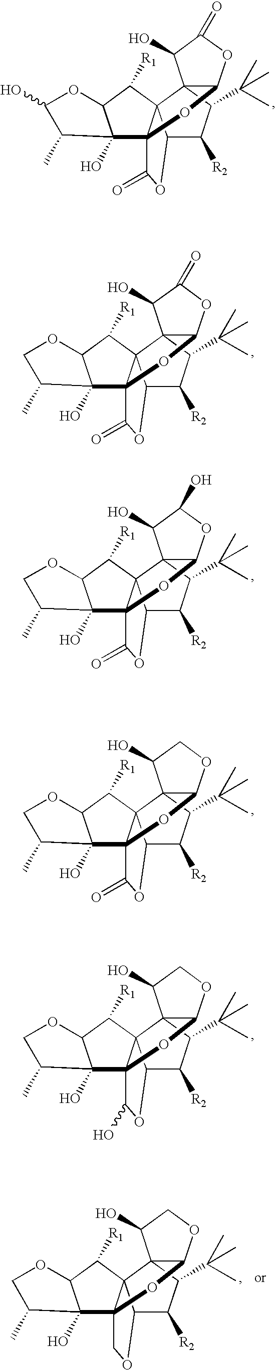 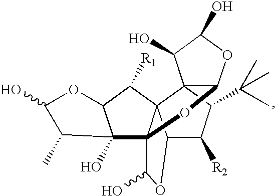
wherein R¹ and R² are, independently, H or OH.

33. The process of claim 1, wherein the process produces a compound having one of the following structures:

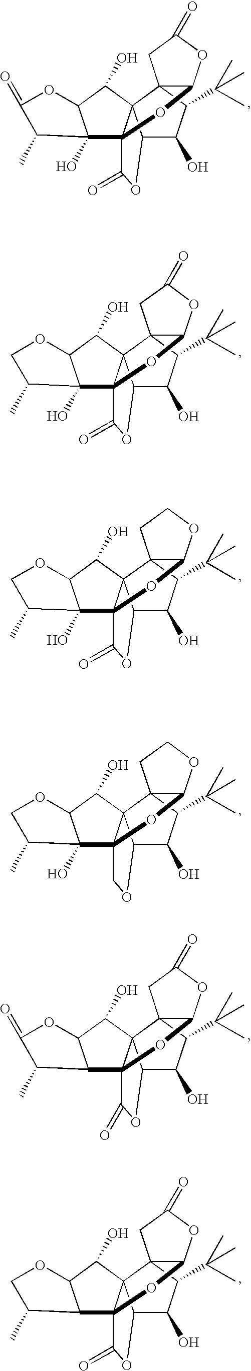

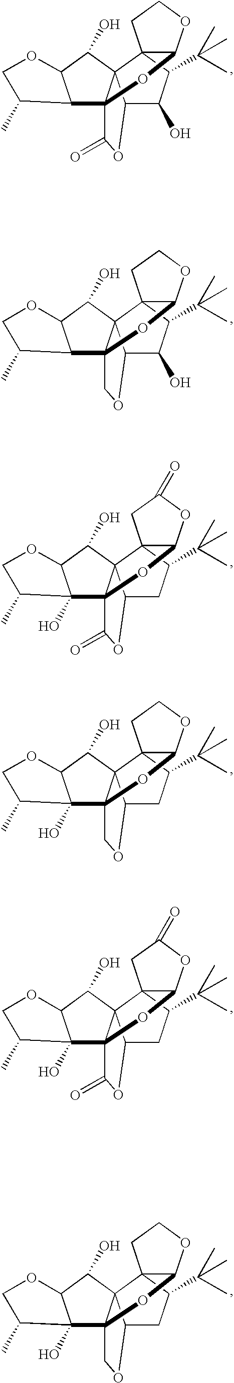

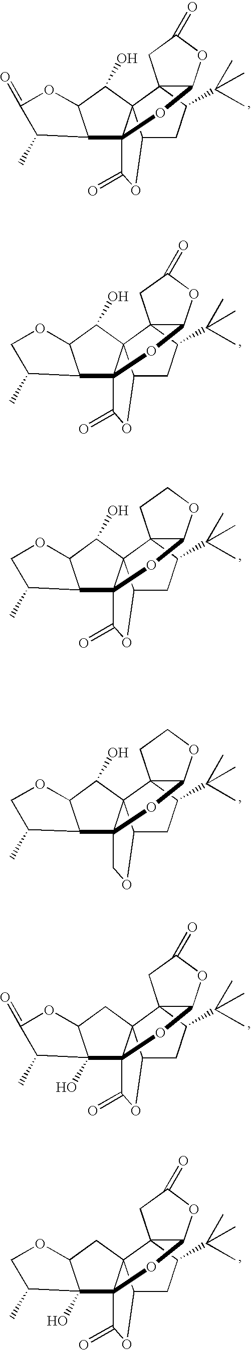

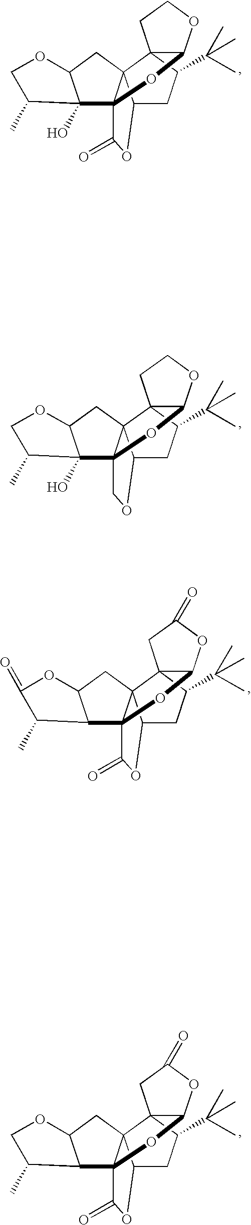

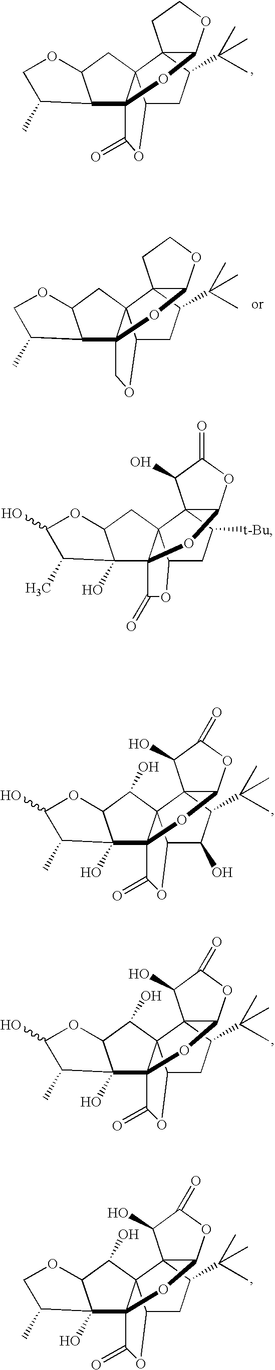

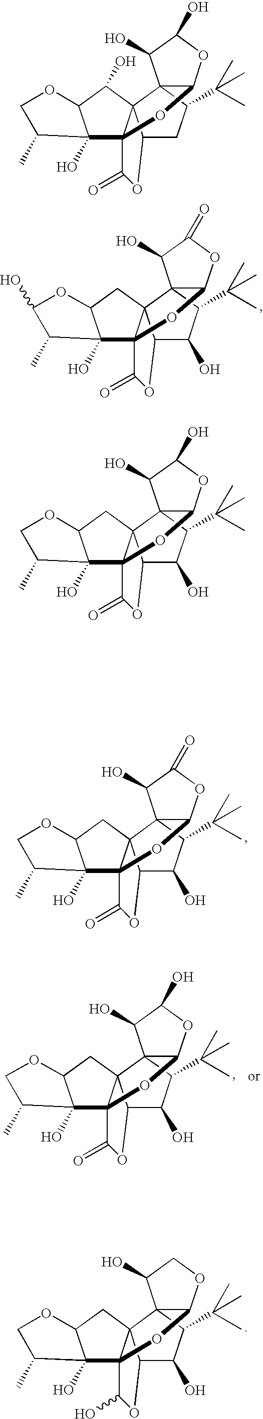

34-43. (canceled)

44. The process of claim 41, wherein the terpene trilactone is a 10-benzyl-ginkgolide or a 10-methyl-ginkgolide and has the structure: 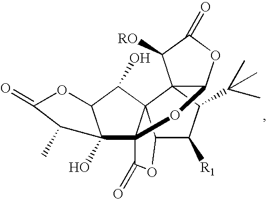
wherein R is Bn or Me and R₁ is H or OH.

45. (canceled)

46. (canceled)

47. The process of claim 1, wherein the allylating agent has the structure:

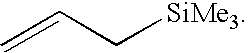

48-52. (canceled)

53. A process for making ginkgolide J from ginkgolide C comprising:
a) exposing the ginkgolide C to a compound having the following structure: 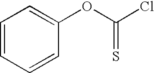
in the presence of DMAP and a suitable solvent so as to make a product having the structure: 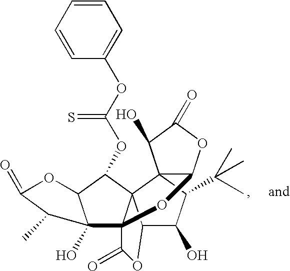
b) exposing the product of step (a) to Et₃SiH and Bz₂0 or Bu₃SnH and AlBN, in the presence of a suitable solvent, and refluxing to produce ginkgolide J;
or
a process for making a ginkgolide triether from a ginkgolide A or ginkgolide J comprising:
a) exposing the ginkgolide to a suitable reducing agent in a suitable solvent so as to so as to reduce lactones of the terpene trilactone to lactols; and
b) exposing the product of step a) to Et₃SiH and BF₃-Et₂0 in a suitable solvent for sufficient time to deoxygenate the lactols to cyclic ethers so as to thereby make the ginkgolide triether;
or
a process of producing ginkgolide M comprising:
(a) exposing 10-benzyl-ginkgolide C or 10-methyl-ginkgolide C to pyridine and (diethylamino)sulfur trifluoride in the presence of a suitable solvent so as to produce a compound having the structure: 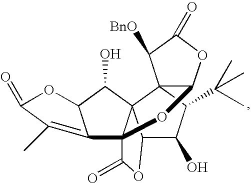
(b) exposing the product of step (a) to H₂ under pressure in the presence of Pd/C so as to produce 14-epi-ginkgolide M having the structure: 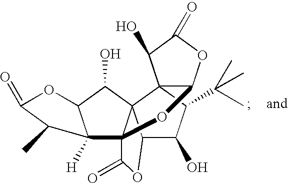
(c) exposing the 14-epi-ginkgolide M of step (b) to DMAP in a suitable solvent for a time sufficient to produce ginkgolide M.

54-69. (canceled)

70. A process for producing a 10-substituted ginkgolide derivative comprising:
(1) exposing a ginkgolide having a hydroxyl group at the 10-position to a compound having the structure: 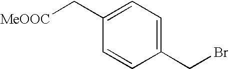
in the presence of a suitable base and a suitable solvent for a time sufficient to produce the 10-substituted ginkgolide derivative; or
(2) exposing a ginkgolide having a hydroxyl group at the 10-position to MeI in the presence of a suitable base and a suitable solvent for a time sufficient to produce the 10-substituted ginkgolide derivative;
or
a process for producing a 7-substituted ginkgolide derivative comprising exposing a ginkgolide having a hydroxyl group at the 7-position to a compound having the structure: 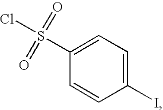
in the presence of a suitable base and a suitable solvent for a time sufficient to produce the 7-substituted ginkgolide derivative;
or
a process of functionalizing a terpene trilactone cage skeleton at a C1, C7, or C10 position comprising exposing the terpene trilactone cage skeleton to an alkylating agent capable of undergoing a subsequent deoxygenation, in the presence of DMAP and a suitable solvent to form the functionalized terpene trilactone cage skeleton.

71-84. (canceled)

85. A process for producing a 7-substituted ginkgolide derivative comprising exposing a ginkgolide having a hydroxyl group at the 7-position to a compound having the structure: 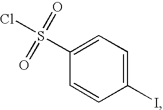
in the presence of a suitable base and a suitable solvent for a time sufficient to produce the 7-substituted ginkgolide derivative;
or
a process for methylating a C10 hydroxyl and/or a C3 hydroxyl of hydroxyl bearing terpene trilactone cage skeleton comprising exposing the terpene trilactone cage skeleton to MeI and KH in a suitable solvent for a sufficient time to methylate the C10 hydroxyl and/or the C3 hydroxyl of the terpene trilactone cage skeleton
or
a process for methylating a C₁₀ hydroxyl and a C3 hydroxyl of a ginkgolide triether comprising exposing the ginkgolide triether to MeI, AgOTf, and Et₃N in a suitable solvent and refluxing to methylate the C10 hydroxyl and the C3 hydroxyl of the ginkgolide triether;
or
a process of replacing a hydroxyl group on a terpene trilactone cage skeleton or a bilobalide comprising exposing a hydroxyl bearing terpene trilactone cage skeleton or bilobalide to an alkylating agent capable of undergoing a subsequent deoxygenation, in the presence of a base and a first suitable solvent to form a first product, and exposing the first product to Bu₃SnH and AlBN in the presence of a second suitable solvent for a time sufficient to deoxygenate the hydroxyl group, so as to thereby replace the hydroxyl group from the terpene trilactone cage skeleton or bilobalide;
or
a process of producing ginkgolide J comprising exposing ginkgolide C to an alkylating agent capable of undergoing a subsequent deoxygenation, in the presence of a base and a first suitable solvent to form a first product, and exposing the first product to Bu₃SnH and AlBN in the presence of a second suitable solvent for a time sufficient to deoxygenate a C1 hydroxyl group of the ginkgolide C, so as to thereby produce ginkgolide J;
or
a process for double dehydrating a ginkgolide comprising exposing the ginkgolide to pyridine and SOCl₂;
or
a process for making ginkgolide L from ginkgolide A comprising exposing the ginkgolide A to (diethylamino)sulfur trifluoride in the presence of a suitable solvent for a time sufficient to produce ginkgolide L;
or
a process of making a compound having the structure: 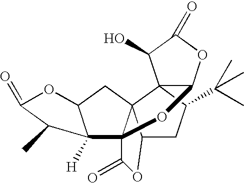
comprising exposing a compound having the structure 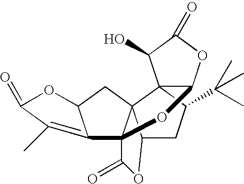
to H₂ under pressure in the presence of Pd/C so as to produce the compound;
or
a process of increasing the hydrophobicity of a lactone bearing terpene trilactone cage skeleton comprising reducing one or more lactones of the lactone bearing terpene trilactone by exposing it to DIBAL-H.

86-90. (canceled)

91. A compound having the following structure: 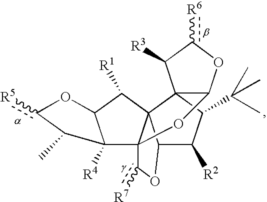
wherein each of R¹, R², and R⁴ is, independently, H or OH;
each of R⁵, R⁶ and R⁷ is H or OH, or O and the respective bond α, β, or γ is present; and
R³ is H, or
R³ is OH when R¹ is H, R² is OH and R⁴ is H,
or when at least one of R⁵, R⁶ and R⁷ is OH,
or when R⁵ is H, R⁶ is O and bond β is present and R⁷ is H,
wherein R⁵ is H or OH when only one of R⁶ or R⁷ is O;
or
a compound having the structure: 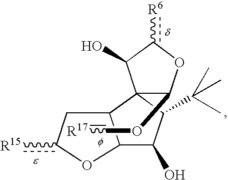
wherein one of R¹⁵, R¹⁶, or R¹⁷ is H or OH, and wherein when R¹⁵, R¹⁶, or R¹⁷ is O, the respective bond δ, ε, or φ is present
or
a compound having the following structure: 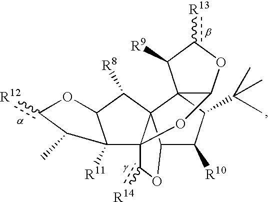
wherein each of R⁸, R⁹ and R¹¹ are, independently, H, OH, OMe or 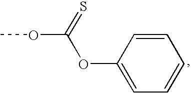
with the proviso that at least two of R⁸, R⁹ and R¹¹ are Ome or at least one of R⁸, R⁹ and R¹¹ is 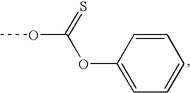
and each of R¹², R¹³ and R¹⁴ is H or OH, or O and the respective bond α, β, or γ is present,
and R¹⁰ is H or OH;
or
a compound having one of the following structures: 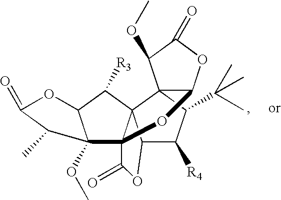 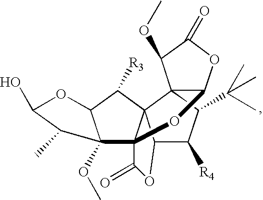
wherein R₃ and R₄ are, independently, H or OMe.

92-110. (canceled)

111. A method of determining whether a test compound is a platelet-activating receptor (PAF) receptor antagonist or agonist comprising:
a) quantitating the activity of a PAF receptor in a PAF receptor-containing membrane or tissue in the presence of a predetermined amount of a PAF receptor agonist;
b) exposing the PAF receptor to a predetermined amount of a compound of claim 91;
c) quantitating the reduction of the PAF receptor activity in the presence of both the predetermined amount of PAF receptor agonist and the predetermined amount of the compound of claim 91; and
d) exposing the PAF receptor to the test compound and quantitating the reduction or increase of the PAF receptor activity in the presence of the test compound as compared to the PAF receptor activity quantitated in step c),
whereby an increase in PAF receptor activity quantitated in step d) as compared to step c) indicates that the test compound is a PAF receptor agonist, and whereby a decrease in PAF receptor activity quantitated in step d) as compared to step c) indicates that the test compound is a PAF receptor antagonist;
or
a method of determining whether a test compound relieves or enhances impairment of long-term potentiation (LTP) by a beta amyloid comprising:
a) quantifying a LTP in a mammalian brain portion;
b) exposing the mammalian brain portion to a predetermined amount of the beta amyloid and quantifying the impairment of the LTP in the mammalian brain portion in the presence of the beta amyloid;
c) exposing the brain to a predetermined amount of a compound of claim 91 sufficient to reduce the impairment of the LTP in the mammalian brain portion by the beta amyloid; and
d) exposing the brain to the test compound and quantitating the reduction or increase of the LTP in the mammalian brain portion in the presence of the test compound as compared to the LTP quantitated in step c),
whereby an increase in LTP quantitated in step d) as compared to step c) indicates that the test compound relieves impairment of LTP by beta amyloid, and whereby a decrease in LTP quantitated in step d) as compared to step c) indicates that the test compound enhances beta-amyloid impairment of LTP;
or
a method of determining whether a test compound inhibits neuronal cell death comprising:
a) exposing a first plurality of neuronal cells to a compound of claim 91;
b) exposing the first plurality of neuronal cells from step a) to a predetermined amount of beta amyloid;
c) determining the rate of neuronal cell death of the first plurality of neuronal cells at a predetermined time after step b)
d) exposing a second plurality of the neuronal cells to the test compound;
e) exposing the second plurality of the neuronal cells from step d) to the predetermined amount of beta amyloid;
f) determining the rate of neuronal cell death of the second plurality of the neuronal cells at a predetermined time after step e); and comparing the rate of neuronal cell death determined in step f) to that determined in step c),
whereby a lower rate of neuronal cell death determined in step f) as compared to step c) indicates that the test compound inhibits neuronal cell death.

112-150. (canceled)

151. A compound having the structure: 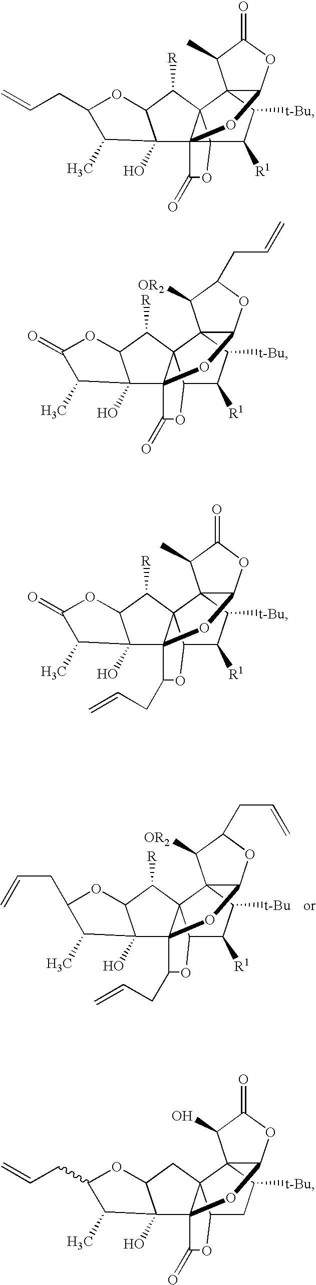
wherein R, R₁ and R₂ are, independently, H, OH, an alkyl, an aryl or a functional group;
or

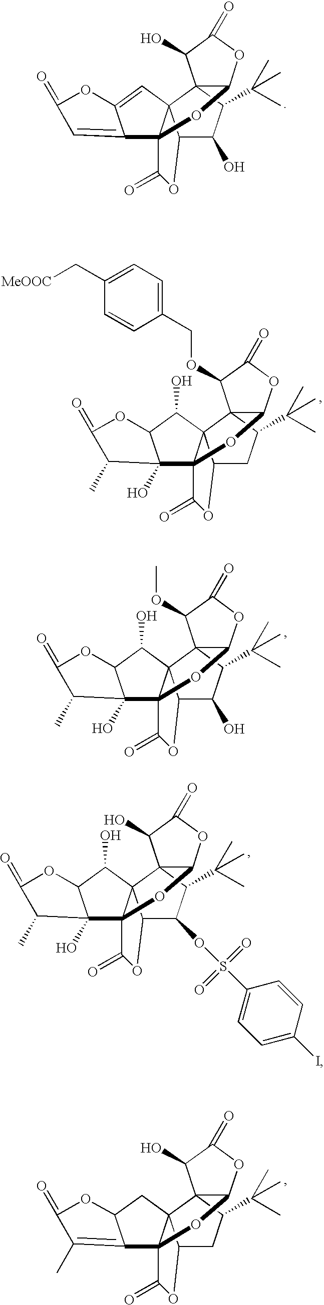

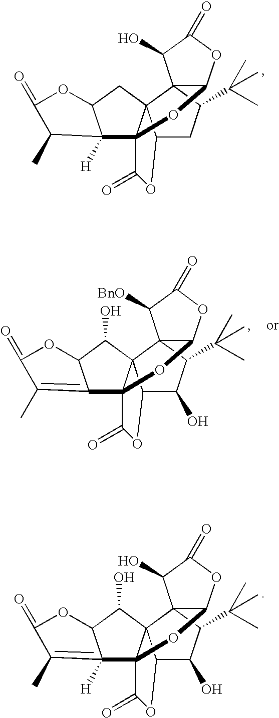

152-165. (canceled)
